(19) United States
(12) Patent Application Publication
(10) Pub. No.: US 2024/0238314 A1
(43) Pub. Date: Jul. 18, 2024

(54) TREATMENT OF ESSENTIAL TREMOR

(71) Applicant: Sage Therapeutics, Inc., Cambridge, MA (US)

(72) Inventors: Helen Anne Colquhoun, Arlington, MA (US); Oluwakemi Bankole, Cobham (GB); Adi Eldar-Lissai, Brookline, MA (US); Margaret Elizabeth Gerbasi, Somerville, MA (US); Jeffrey A. Wald, Cary, NC (US)

(21) Appl. No.: 18/554,250

(22) PCT Filed: Apr. 11, 2022
(86) PCT No.: PCT/US2022/024264

§ 371 Date: Oct. 6, 2023

Related U.S. Application Data
(60) Provisional application No. 63/173,867, filed on Apr. 12, 2021.

(51) Int. Cl. A61K 31/58; A61K 9/28; A61P 25/14

(52) U.S. Cl. CPC A61K 31/58; A61K 9/28; A61P 25/14

(57) ABSTRACT

The present invention provides a method of treating essential tremor in a subject in need thereof comprising administering Compound 1

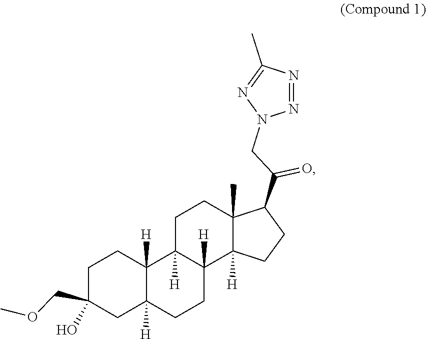

or a pharmaceutically acceptable salt thereof, or a pharmaceutical composition comprising Compound 1 or a pharmaceutically acceptable salt thereof, wherein the subject experiences (a) a reduction in tremor amplitude of at least about 5% following treatment onset, (b) a reduction in TETRAS activities of daily living (ADL) subscale total score following treatment onset, or (c) both.

### CROSS REFERENCE TO RELATED APPLICATION

This PCT application claims the benefit of U.S. provisional application No. 63/173,867, filed on 12 Apr. 2021, the entire content of which is hereby incorporate by reference in its entirety.

### FIELD OF THE INVENTION

The present invention relates to compounds and methods of treating essential tremor in a subject in need thereof.

### BACKGROUND

Progesterone and its metabolites have been demonstrated to have profound effects on brain excitability (Backstrom, T. et al., Acta Obstet. Gynecol. Scand. Suppl. 130:19-24 (1985); Pfaff, D. W and McEwen, B. S., Science 219:808-814 (1983); Gyermek et al., J Med Chem. 11: 117 (1968); Lambert, J. et al., Trends Pharmacol. Sci. 8:224-227 (1987)). Additionally, several lines of evidence suggest that cerebellar dysfunction through the cerebello-thalamocortical pathway play a key role in essential tremor (ET) (McAuley 2000; Pinto 2003; Elble 2009; Schnitzler 2009; Deuschl 2009). Activation studies with positron emission tomography (PET) indicate abnormally increased regional cerebral blood flow in the cerebellus both at rest and when tremor is provoked by unilateral arm extension (Boechker 1994, Wills 1996). Post-mortem analysis revealed a 35% reduction of GABA_{A} receptors and a 22-31% reduction of GABA_{B} receptors in the dentate nucleus of cerebella from ET patients (Paris-Robidas 2012).

There is increasing evidence to support the use of neuroactive steroids, e.g., a neuroactive steroid as described herein, in the treatment and prevention of tremor (e.g., essential tremor).

### SUMMARY OF THE INVENTION

The present invention provides a method of treating essential tremor in a subject in need thereof, comprising administering to the subject Compound 1

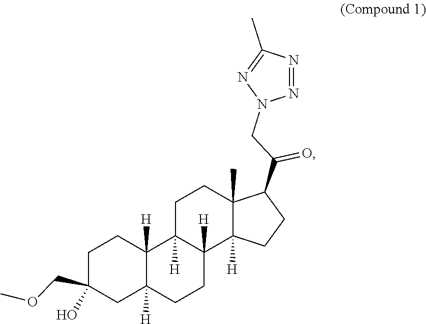

or a pharmaceutically acceptable salt thereof.

In one aspect, the present invention provides a method of treating essential tremor in a subject in need thereof, comprising administering to the subject a dose of from about 5 mg to about 80 mg amount of Compound 1

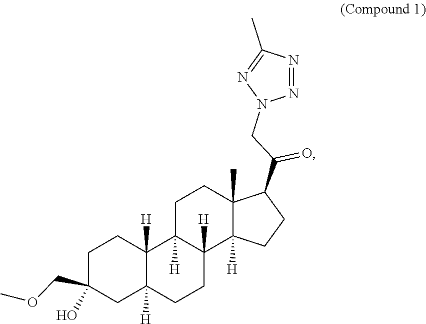

or a dose equivalent of a pharmaceutically acceptable salt thereof, at least once per day.

In some implementations, the subject is administered a dose of from about 35 mg to about 70 mg of Compound 1 or a dose equivalent of a pharmaceutically acceptable salt thereof at least once per day (e.g., QD, BID, TID, or QID). For instance, the subject is administered a dose of from about 30 mg to about 60 mg of Compound 1 or a dose equivalent of a pharmaceutically acceptable salt thereof at least once per day (e.g., QD, BID, TID, or QID).

In some implementations, the subject is administered a dose of from about 10 mg to about 35 mg of Compound 1 or a dose equivalent of a pharmaceutically acceptable salt thereof at least once per day (e.g., QD, BID, TID, or QID). For instance, the subject is administered a dose of from about 15 mg to about 30 mg of Compound 1 or a dose equivalent of a pharmaceutically acceptable salt thereof at least once per day (e.g., QD, BID, TID, or QID).

In some implementations, the subject is administered a dose of from about 10 mg to about 20 mg of Compound 1 or a dose equivalent of a pharmaceutically acceptable salt thereof at least once per day (e.g., QD, BID, TID, or QID).

In some implementations, the subject is administered a dose of from about 25 mg to about 35 mg of Compound 1 or a dose equivalent of a pharmaceutically acceptable salt thereof at least once per day (e.g., QD, BID, TID, or QID).

In some implementations, the subject is administered a dose of from about 40 mg to about 50 mg of Compound 1 or a dose equivalent of a pharmaceutically acceptable salt thereof at least once per day (e.g., QD, BID, TID, or QID).

In some implementations, the subject is administered a dose of from about 55 mg to about 65 mg of Compound 1 or a dose equivalent of a pharmaceutically acceptable salt thereof at least once per day (e.g., QD, BID, TID, or QID).

In some implementations, such as any of the implementations described herein, wherein Compound 1 or a dose equivalent or a pharmaceutically acceptable salt thereof is administered to the subject once per day, the administration is at night (e.g., before bed).

In one aspect, the present invention provides a method of treating essential tremor in a subject in need thereof, comprising administering to the subject a therapeutically effective amount of Compound 1

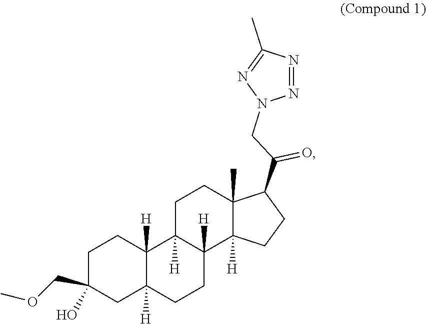

or a pharmaceutically acceptable salt thereof, wherein the subject experiences a reduction in tremor amplitude of at least about 5% following treatment onset.

In some implementations, the subject experiences a reduction in tremor amplitude of at least about 10% following treatment onset. For example, the subject experiences a reduction in tremor amplitude of at least about 12% (e.g., at least about 15%, at least about 20%, or at least about 25%) following treatment onset. For example, the subject experiences a reduction in tremor amplitude of at least about 30% (e.g., at least about 35%, at least about 40%, or at least about 50%) following treatment onset. In other examples, the subject experiences a reduction in tremor amplitude of at least about 50% following treatment onset. In some examples, the subject experiences a reduction in tremor amplitude of at least about 75% following treatment onset. And, in some examples, the subject experiences a reduction in tremor amplitude of from about 30% to about 99% (e.g., from about 30% to about 85%, from about 30% to about 80%, from about 30% to about 75%, and the like).

In some implementations, the subject experiences a reduction in tremor amplitude about 5 days following treatment onset. In some implementations, the subject experiences a reduction in tremor amplitude about 8 days following treatment onset. For example, the subject experiences a reduction in tremor amplitude about 10 days, about 12 days, about 14 days, about 16 days, about 18 days, about 20 days, about 25 days, about 30 days, or about 40 days following treatment onset.

In some implementations, the subject has an initial TETRAS performance subscale part 4 upper limb tremor total score of no greater than 12 prior to treatment onset. In some implementations, the subject has an initial TETRAS performance subscale part 4 upper limb tremor total score of at least about 4 prior to treatment onset. In some implementations, the subject has an initial TETRAS performance subscale part 4 upper limb tremor total score of at least about 6 prior to treatment onset. In some implementations, the subject has an initial TETRAS performance subscale part 4 upper limb tremor total score of at least about 8 prior to treatment onset. In some implementations, the subject has an initial TETRAS performance subscale part 4 upper limb tremor total score of at least about 10 prior to treatment onset. In some implementations, the subject has an initial TETRAS performance subscale part 4 upper limb tremor total score of at least about 12 prior to treatment onset. In some implementations, the subject has an initial TETRAS performance subscale part 4 upper limb tremor total score of at least about 20 prior to treatment onset. In some implementations, the subject has an initial TETRAS performance subscale part 4 upper limb tremor total score of at least about 24 prior to treatment onset. In some implementations, the subject has an initial TETRAS performance subscale part 4 upper limb tremor total score of at least about 28 prior to treatment onset.

Another aspect of the present invention provides a method of treating essential tremor in a subject in need thereof, comprising administering to the subject a therapeutically effective amount of Compound 1

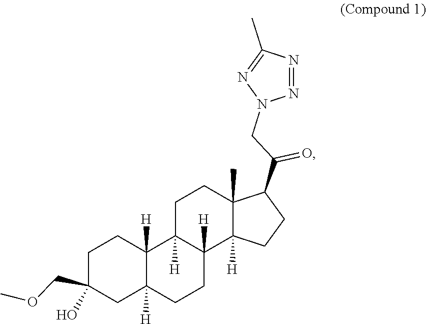

or a pharmaceutically acceptable salt thereof, wherein the subject experiences a reduction in TETRAS activities of daily living (ADL) subscale total score following treatment onset.

In some implementations, the subject experiences a reduction in TETRAS ADL subscale total score of at least about 5% following treatment onset. For example, the subject experiences a reduction in TETRAS ADL subscale total score of at least about 10% following treatment onset. In other examples, the subject experiences a reduction in TETRAS ADL subscale total score of at least about 15% following treatment onset. In other examples, the subject experiences a reduction in TETRAS ADL subscale total score of at least about 20% following treatment onset.

In some implementations, the subject experiences the reduction in TETRAS ADL subscale total score about 8 days following treatment onset.

In some implementations, the subject experiences a reduction in TETRAS ADL subscale total score about 10 days following treatment onset.

In some implementations, the subject experiences a reduction in TETRAS ADL subscale total score about 15 days following treatment onset.

In some implementations, the subject experiences a reduction in TETRAS ADL subscale total score about 20 days following treatment onset.

In some implementations, the subject experiences a reduction in TETRAS ADL subscale total score about 25 days following treatment onset.

In some implementations, the subject experiences a reduction in TETRAS ADL subscale total score about 30 days following treatment onset.

In some implementations, the subject has an initial TETRAS ADL subscale total score of at least 12 prior to treatment onset.

In some implementations, the subject has an initial TETRAS ADL subscale total score of less than about 28 prior to treatment onset.

In some implementations, the subject has an initial TETRAS ADL subscale total score of at least about 20 prior to treatment onset.

In some implementations, the subject has an initial TETRAS ADL subscale total score of at least about 24 prior to treatment onset.

In some implementations, the subject has an initial TETRAS ADL subscale total score of at least about 28 prior to treatment onset.

In some implementations, the subject is administered a dose of from about 5 mg to about 80 mg of Compound 1 or a dose equivalent of a pharmaceutically acceptable salt thereof, at least once per day. For example, the subject is administered a dose of from about 10 mg to about 70 mg of Compound 1 or a dose equivalent of a pharmaceutically acceptable salt thereof, at least once per day. In other examples, the subject is administered a dose of from about 50 mg to about 70 mg of Compound 1 or a dose equivalent of a pharmaceutically acceptable salt thereof, at least once per day. In some examples, the subject is administered a dose of from about 10 mg to about 30 mg of Compound 1 or a dose equivalent of a pharmaceutically acceptable salt thereof, at least once per day. And, in some examples, the subject is administered a dose of about 10 mg, about 15 mg, about 30 mg, about 45 mg, or about 60 mg of Compound 1 or a dose equivalent of a pharmaceutically acceptable salt thereof, at least once per day.

In some implementations, the subject is administered a dose of about 60 mg of Compound 1 or a dose equivalent of a pharmaceutically acceptable salt thereof, at least once per day.

In some implementations, the subject is administered a dose of from about 30 mg to about 50 mg of Compound 1 or a dose equivalent of a pharmaceutically acceptable salt thereof, at least once per day.

In some implementations, the subject is administered a dose of about 45 mg of Compound 1 or a dose equivalent of a pharmaceutically acceptable salt thereof, at least once per day.

In some implementations, the subject is administered a dose of from about 20 mg to about 40 mg of Compound 1 or a dose equivalent of a pharmaceutically acceptable salt thereof, at least once per day.

In some implementations, Compound 1, or the pharmaceutically acceptable salt thereof, is administered once per day.

In some implementations, Compound 1, or a pharmaceutically acceptable salt thereof, is administered once per day for at least about 28 days.

In some implementations, Compound 1, or a pharmaceutically acceptable salt thereof, is administered once per day chronically.

In some implementations, the subject has a plasma concentration of Compound 1 of at least about 175 ng/mL about 8 days following treatment onset. For example, the subject has a plasma concentration of Compound 1 of about 200 ng/mL about 8 days following treatment onset. In other examples, the subject has a plasma concentration of Compound 1 of from about 200 ng/mL to about 250 ng/mL about 8 days following treatment onset. In some examples, the subject has a plasma concentration of Compound 1 of at least about 210 ng/mL about 15 days following treatment onset. In some examples, the subject has a plasma concentration of Compound 1 of from about 210 ng/mL to about 450 ng/mL about 15 days following treatment onset. In another example, the subject has a plasma concentration of Compound 1 of at least about 230 ng/mL about 22 days following treatment onset. In some examples, the subject has a plasma concentration of Compound 1 of from about 230 ng/mL to about 390 ng/mL about 22 days following treatment onset. And, in some examples, the subject has a plasma concentration of Compound 1 of greater than about 250 ng/mL about 29 days following treatment onset.

In some implementations, Compound 1, or the pharmaceutically acceptable salt thereof, is administered orally. For example, Compound 1, or the pharmaceutically acceptable salt thereof, is administered as a tablet or capsule.

Another aspect of the present invention provides a method of treating essential tremor in a subject in need thereof, comprising administering to the subject an initial dose of from about 5 mg to about 80 mg of Compound 1

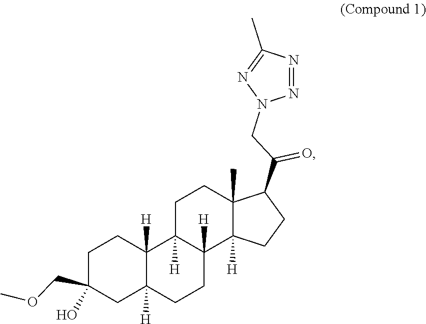

or a dose equivalent of a pharmaceutically acceptable salt thereof, wherein the initial dose is administered at least once per day (e.g., at night (e.g., at bedtime)).

In some implementations, the initial dose is administered from onset of treatment for a duration of from about 1 to about 30 days. For example, the initial dose is administered from onset of treatment for a duration of about 28 days.

In some implementations, the initial dose is from about 55 mg to about 70 mg of Compound 1 or a dose equivalent of a pharmaceutically acceptable salt thereof.

In some implementations, the initial dose is administered once per day.

Some implementations comprise administering to the subject a second dose comprising from about 25 mg to about 50 mg of Compound 1, or a dose equivalent of a pharmaceutically acceptable salt thereof, wherein the second dose is administered on the day following the last administration of the initial dose. In some examples, the second dose comprises from about 30 mg to about 45 mg of Compound 1, or a dose equivalent of a pharmaceutically acceptable salt thereof. In other examples, the second dose comprises from about 25 mg to about 35 mg of Compound 1, or a dose equivalent of a pharmaceutically acceptable salt thereof.

In some implementations, the second dose is administered for a duration of about 60 days or less. In other implementations, the second dose is administered for a duration of greater than about 60 days.

In some implementations, the second dose is administered at least once per day. For instance, the second dose is administered once per day.

Some implementations comprise administering to the subject a third dose comprising from about 5 mg to about 24 mg of Compound 1, or a dose equivalent of a pharmaceutically acceptable salt thereof, wherein the third dose is administered on the day following the last administration of the second dose. In some examples, the third dose comprises from about 5 mg to about 20 mg of Compound 1, or a dose equivalent of a pharmaceutically acceptable salt thereof. In other examples, the third dose comprises from about 7.5 mg to about 17.5 mg of Compound 1, or a dose equivalent of a pharmaceutically acceptable salt thereof.

In some implementations, the third dose is administered at least once per day. For instance, the third dose is administered once per day.

In some implementations, Compound 1, or the pharmaceutically acceptable salt thereof, is administered orally. For example, Compound 1, or the pharmaceutically acceptable salt thereof, is administered as a tablet or capsule.

Another aspect of the present invention provides a method of treating essential tremor in a subject in need thereof, comprising administering to the subject a therapeutically effective amount of Compound 1

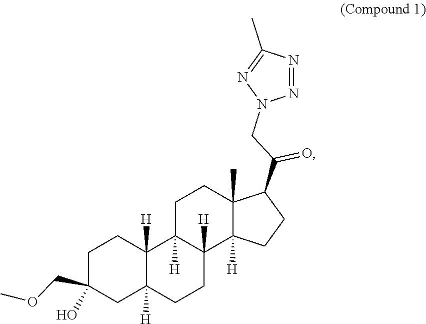

or a pharmaceutically acceptable salt thereof, wherein the subject has a plasma concentration of Compound 1 of from about 100 ng/mL to about 450 ng/mL about 8 days following treatment onset.

In some implementations, the subject has a plasma concentration of Compound 1 of from about 150 ng/mL to about 400 ng/mL about 8 days following treatment onset. For example, the subject has a plasma concentration of Compound 1 of from about 175 ng/mL to about 400 ng/mL about 8 days following treatment onset. In other examples, the subject has a plasma concentration of Compound 1 of from about 175 ng/mL to about 250 ng/mL about 8 days following treatment onset. In other examples, the subject has a plasma concentration of Compound 1 of from about 200 ng/mL to about 250 ng/mL about 8 days following treatment onset. In some examples, the plasma concentration as described herein is the plasma concentration, C_{trough}.

Another aspect of the present invention provides a method of treating essential tremor in a subject in need thereof, comprising administering to the subject a therapeutically effective amount of Compound 1

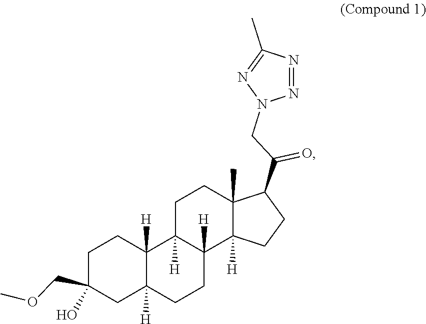

or a pharmaceutically acceptable salt thereof, wherein the subject has a plasma concentration of Compound 1 of from about 210 ng/mL to about 475 ng/mL about 15 days following treatment onset.

In some implementations, the subject has a plasma concentration of Compound 1 of from about 210 ng/mL to about 450 ng/mL about 15 days following treatment onset. For example, the subject has a plasma concentration of Compound 1 of from about 210 ng/mL to about 350 ng/mL about 15 days following treatment onset. For example, the subject has a plasma concentration of the compound of Compound 1 of from about 210 ng/mL to about 280 ng/mL about 15 days following treatment onset. In other examples, the subject has a plasma concentration of Compound 1 of from about 320 ng/mL to about 420 ng/mL about 15 days following treatment onset. And, in some examples, the subject has a plasma concentration of Compound 1 of from about 275 ng/mL to about 490 ng/mL about 15 days following treatment onset. In some examples, the plasma concentration as described herein is the plasma concentration, C_{trough}.

Another aspect of the present invention provides a method of treating essential tremor in a subject in need thereof, comprising administering to the subject a therapeutically effective amount of Compound 1

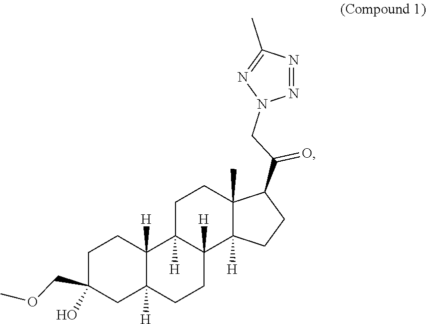

or a pharmaceutically acceptable salt thereof, wherein the subject has a plasma concentration of Compound 1 of from about 230 ng/mL to about 390 ng/mL at least about 22 days following treatment onset. In some examples, the plasma concentration as described herein is the plasma concentration, C_{trough}.

### BRIEF DESCRIPTION OF THE DRAWINGS

The figures below are provided by way of example and are not intended to limit the scope of the claimed invention.

FIG. 1 is a diagram of subject disposition according to Example 1.

FIG. 2 is a plot of LS Mean change from baseline in The Essential Tremor Rating Assessment Scale (TETRAS) performance subscale part 4 upper limb tremor total score over time by treatment group (Full Analysis Set) according to Example 1.

FIG. 3 is a forest plot of change from baseline in TETRAS performance subscale part 4 upper limb total score at day 29 (Full Analysis Set) according to Example 1.

FIG. 4 is a plot of LS Mean change from baseline in TETRAS performance subscale part 4 upper limb tremor total score over time by treatment group (For those patients having a baseline TETRAS performance subscale part 4 upper limb total score ≥12) according to Example 1.

FIG. 5 is a plot of LS Mean change from baseline in TETRAS performance subscale part 4 upper limb tremor total score over time by treatment group (Per-Protocol Set) according to Example 1.

FIG. 6 is a plot of LS Mean change from baseline in TETRAS ADL total score over time by treatment group (Full Analysis Set) according to Example 1.

FIG. 7 is a forest plot of change from baseline in TETRAS ADL total score at day 29 (Full Analysis Set) according to Example 1.

FIG. 8 is a plot of LS Mean change from baseline in TETRAS performance subscale total score over time by treatment group (Full Analysis Set) according to Example 1.

FIG. 9 is a forest plot of change from baseline in TETRAS performance subscale total score at day 29 (Full Analysis Set) according to Example 1.

FIG. 10 is a plot of LS Mean change from baseline in TETRAS performance subscale part 4/6/7/8 total score over time by treatment group (Full Analysis Set) according to Example 1.

FIG. 11 is a forest plot of change from baseline in TETRAS performance subscale part 4/6/7/8 total score at day 29 (Full Analysis Set) according to Example 1.

### DETAILED DESCRIPTION

The present invention provides a method of treating essential tremor, comprising the administration of a therapeutically effective amount of Compound 1

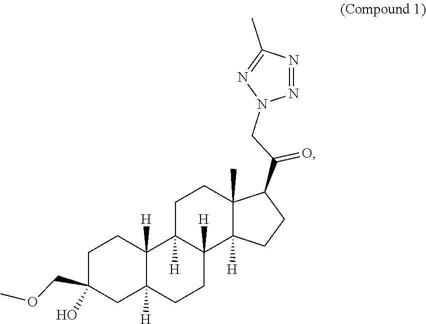

or a pharmaceutically acceptable salt thereof to a subject (e.g., human) in need thereof.

### I. DEFINITIONS

As used herein, the term “modulation” refers to the inhibition or potentiation of GABA_{A} receptor function. A “modulator” (e.g., a compound or pharmaceutically acceptable salt thereof that modulates GABA_{A} receptor function) may be, for example, an agonist, partial agonist, antagonist, or partial antagonist of the GABA_{A} receptor.

As used herein, the term “about”, when referring to a numerical value or range of values, allows for a degree of variability in the value or range or values, for example, within 10%, or within 5% of a stated value or of a stated limit of a range.

As used herein, the term “pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

As used herein, the term “pharmaceutically acceptable salt” refers to a salt of a compound of the invention that is pharmaceutically acceptable and that possesses the desired pharmacological activity of the parent compound. In particular, such salts are non-toxic, and may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counterion of an acidic functional group. Such cations are exemplified by sodium, potassium, calcium, magnesium, ammonium, tetraalkylammonium cations, and the like. See, e.g., Berge, et al., J. Pharm. Sci. (1977) 66(1): 1-79.

As used herein, the term “prodrug” is intended to encompass therapeutically inactive compounds that, under physiological conditions, are converted into the therapeutically active agents of the present invention. One method for making a prodrug is to design selected moieties that are hydrolyzed or cleaved at a targeted in vivo site of action under physiological conditions to reveal the desired molecule which then produces its therapeutic effect. In certain embodiments, the prodrug is converted by an enzymatic activity of the subject.

In an alternate embodiment, the present invention provides prodrugs of compounds described herein, wherein the prodrug includes a cleavable moiety on the C3 hydroxy as depicted in Formulae depicted herein (e.g., Compound 1).

As used herein, the term “tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro- forms of phenylnitromethane that are likewise formed by treatment with acid or base. Tautomeric forms may be relevant to the attainment of the optimal chemical reactivity and biological activity of a compound of interest.

As used herein, the term “subject” to which administration is contemplated includes, but is not limited to, humans (e.g., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a mammal. In other embodiments, the subject is a human.

As used herein, the terms “disease”, “disorder”, and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition (or any symptom thereof), or retards or slows the progression of the disease, disorder or condition (“therapeutic treatment”), and also contemplates a prophylactic action that occurs before a subject begins to suffer from the specified disease, disorder or condition.

In general, the “effective amount” of a compound refers to an amount sufficient to elicit the desired biological response, e.g., to treat a CNS-related disorder, or is sufficient to induce anesthesia or sedation. As will be appreciated by those of ordinary skill in the art, the effective amount of a compound of the invention may vary depending on such factors as the desired biological endpoint, the pharmacokinetics of the compound, the disease being treated, the mode of administration, and the age, weight, health, and condition of the subject.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

In an alternate embodiment, the present invention contemplates administration of the compounds of the present invention or a pharmaceutically acceptable salt or a pharmaceutically acceptable composition thereof, as a prophylactic before a subject begins to suffer from the specified disease, disorder or condition. As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

As used herein, the term “treatment onset” and “onset of treatment” are used interchangeably to refer to the day in which a treatment (e.g., administration of Compound 1 or a pharmaceutically acceptable salt thereof) begins. For example, the day of treatment onset is the day in which a chemotherapy is first administered. For dosage regimes comprising the administration of 2 or more therapies, treatment onset is the day in which Compound 1 or a pharmaceutically acceptable salt thereof is first administered.

As used herein, “The Essential Tremor Rating Assessment Scale (TETRAS) activities of daily living (ADL) subscale total score” refers to an assessment scale for essential tremor developed by The Tremor Research Group (Elble, R. J., J. Neurol. Neuromedicine 1(4):34-38 (2016); Elble, R. J. et. al., Mov. Disord. 27(12); 1567-1569 (2012)). The TETRAS ADL subscale includes 12 metrics or characteristics, wherein each metric or characteristic is scored 0, 1, 2, 3, or 4 based on the severity of the characteristic present in the subject. Scores for each of the 12 characteristics are then added to give a total score of no greater than 48. The 12 characteristics scored in the TETRAS ADL subscale total score include:

### 1) Speaking.

0=Normal. 1=Slight voice tremulousness, only when “nervous”. 2=Mild voice tremor. All words easily understood. 3=Moderate voice tremor. Some words difficult to understand. 4=Severe voice tremor, most words difficult to understand.

2) Feeding with a Spoon.

0=Normal. 1=Slightly abnormal. Tremor is present but does not interfere with feeding with a spoon. 2=Mildly abnormal, spills a little. 3=Moderately abnormal, spills a lot or changes strategy to complete task such as using two hands or leaning over. 4=Severely abnormal, cannot feed with a spoon.

3) Drinking from a Glass.

0=Normal. 1=Slightly abnormal, tremor is present but does not interfere with drinking from a glass. 2=Mildly abnormal, spills a little. 3=Moderately abnormal, spills a lot or changes strategy to complete task such as using two hands or leaning over. 4=Severely abnormal, cannot drink from a glass or uses straw or sippy cup.

### 4) Hygiene.

0=Normal. 1=Slightly abnormal, tremor is present but does not interfere with hygiene. 2=Mildly abnormal, some difficulty but can complete task. 3=Moderately abnormal, unable to do most fine tasks such as putting on lipstick or shaving unless changes strategy such as using two hands or using the less affected hand. 4=Severely abnormal, cannot complete hygiene activities independently.

### 5) Dressing.

0=Normal. 1=Slightly abnormal, tremor is present but does not interfere with dressing. 2=Mildly abnormal, able to do everything but has difficulty due to tremor. 3=Moderately abnormal, unable to do most dressing unless uses strategy such as using Velcro, buttoning shirt before putting it on or avoiding shoes with laces. 4=Severely abnormal, cannot dress independently.

### 6) Pouring.

0=Normal. 1=Slightly abnormal, tremor is present but does not interfere with pouring. 2=Mildly abnormal, must be very careful to avoid spilling but may spill occasionally. 3=Moderately abnormal, must use two hands or uses other strategies to avoid spilling. 4=Severely abnormal, cannot pour.

### 7) Carrying Food Trays, Plates or Similar Items.

0=Normal. 1=Slightly abnormal, tremor is present but does not interfere with carrying food trays, plates or similar items. 2=Mildly abnormal, must be very careful to avoid spilling items on food tray. 3=Moderately abnormal, uses strategies such as holding tightly against body to carry. 4=Severely abnormal, cannot carry food trays or similar items.

### 8) Using Keys.

0=Normal. 1=Slightly abnormal, tremor is present but can insert key with one hand without difficulty. 2=Mildly abnormal, commonly misses target but still routinely puts key in lock with one hand. 3=Moderately abnormal, needs to use two hands or other strategies to put key in lock. 4=Severely abnormal, cannot put key in lock.

### 9) Writing.

0=Normal. 1=Slightly abnormal, tremor present but does not interfere with writing. 2=Mildly abnormal, difficulty writing due to the tremor. 3=Moderately abnormal, cannot write without using strategies such as holding the writing hand with the other hand, holding pen differently or using large pen. 4=Severely abnormal, cannot write.

### 10) Working.

0=Normal. 1=Slightly abnormal, tremor is present but does not affect performance at work or at home. 2=Mildly abnormal, tremor interferes with work; able to do everything, but with errors. 3=Moderately abnormal, unable to continue working without using strategies such as changing jobs or using special equipment. 4=Severely abnormal, cannot perform any job or household work.

11) Overall Disability with Most Affected Task (Name Task, e.g., Using Computer Mouse, Writing, Etc.).

0=Normal. 1=Slightly abnormal, tremor present but does not affect task. 2=Mildly abnormal, tremor interferes with task but is still able to perform task. 3=Moderately abnormal, can do task but must use strategies. 4=Severely abnormal, cannot do the task.

### 12) Social Impact.

0=None. 1=Aware of tremor, but it does not affect lifestyle or professional life. 2=Feels embarrassed by tremor in some social situations or professional meetings. 3=Avoids participating in some social situations or professional meetings because of tremor. 4=Avoids participating in most social situations or professional meetings because of tremor.

As used herein, “The Essential Tremor Rating Assessment Scale (TETRAS) performance subscale total score” refers to an assessment scale for essential tremor developed by The Tremor Research Group (Elble, R. J., J. Neurol. Neuromedicine 1(4):34-38 (2016); Elble, R. J. et. al., Mov. Disord. 27(12); 1567-1569 (2012)). The TETRAS ADL performance subscale includes 9 metrics or characteristics, wherein each metric or characteristic is scored 0 to 4. Some metrics or characteristics are performed on both right and left limbs and/or include multiple subitems. Scores for each of the 9 characteristics are then added to give a total score of no greater than 64. The 9 metrics or characteristics include:

1) Head tremor: The head is rotated fully left and right and then observed for 10 s in mid position. Patient then is instructed to gaze fully to the left and then to the right with the head in mid position. The nose should be used as the landmark to assess and rate the largest amplitude excursions during the examination. 0=no tremor. 1=slight tremor (<0.5 cm). 2=mild tremor (0.5-<2.5 cm). 3=moderate tremor (2.5-5 cm). 4=severe or disfiguring tremor (>5 cm).

2) Face (including jaw) tremor: Smile, close eyes, open mouth, purse lips. The highest amplitude of the most involved facial anatomy is scored, regardless of whether it occurs during rest or activation. Repetitive blinking or eye fluttering should not be considered as part of facial tremor. 0=no tremor. 1=slight; barely perceptible tremor. 2=mild: noticeable tremor. 3=moderate: obvious tremor, present in most voluntary facial contractions. 4=severe: gross disfiguring tremor.

3) Voice tremor: First ask subject to produce an extended “aaah” sound and “eee” sound for 5 seconds each. Then assess speech during normal conversation by asking patients “How do you spend your average day?” 0=no tremor. 1=slight: tremor during “aaah” and “eee” and no tremor during speech. 2=mild: tremor in “aaah” and “eee” and minimal tremor in speech. 3=moderate: obvious tremor in speech that is fully intelligible. 4=severe: some words difficult to understand.

4) Upper limb tremor: Tremor is assessed during three maneuvers: forward horizontal reach posture, lateral “wing beating” posture and finger-nose-finger testing. Each upper limb is assessed and scored individually. The forward horizontal reach posture is held for 5 seconds. The lateral wing beating posture is held for 20 seconds. The finger-nose-finger movement is executed three times. Amplitude assessment should be estimated using the maximally displaced point of the hand at the point of greatest displacement along any single plane. For example, the amplitude of a pure supination-pronation tremor, pivoting around the wrist would be assessed at either the thumb or fifth digit.

Task

Forward outstretched postural tremor: Subjects should bring
their arms forward, slightly lateral to midline and parallel to the
ground for 5 seconds. The wrist should also be straight and the
fingers abducted so that they do not touch each other. Each upper
limb is assessed and scored individually.
Lateral “wing beating” postural tremor: Subjects will abduct
their arms parallel to the ground and flex the elbows so that the
two hands do not quite touch each other and are at the level of the
nose. The fingers are abducted so that they do not touch each
other. The posture should be held for 20 seconds. Each upper
limb is assessed and scored individually.
Kinetic tremor: Subjects extend only their index finger. They
then touch a set object or the examiners finger located to the full
extent of their reach, which is located at the same height (parallel
to the ground) and slightly lateral to the midline. Subjects then
touch their own nose (or chin if the tremor is severe) and repeat
this back and forth three times. Only the position along the
trajectory of greatest tremor amplitude is assessed. This will
typically be either at the nose or at the point of full limb
extension. Each upper limb is assessed and scored individually.

For all three hand tremor ratings: 0=no tremor. 1=tremor is barely visible. 1.5=tremor is visible, but less than 1 cm. 2=tremor is 1-<3 cm amplitude. 2.5=tremor is 3-<5 cm amplitude. 3=tremor is 5-<10 cm amplitude. 3.5=tremor is 10-<20 cm amplitude. 4=tremor is >20 cm amplitude.

5) Lower limb tremor: Raise each lower limb horizontally parallel to the ground for 5 seconds. Then perform a standard heel to shin maneuver with each leg, three times. The maximum tremor in either maneuver is scored, and only the limb with the largest tremor is scored. Tremor may exist in any part of the limb, including foot. 0=no tremor. 1=slight: barely perceptible. 2=mild, less than 1 cm at any point. 3=moderate tremor, less than 5 cm at any point. 4=severe tremor, greater than 5 cm.

6) Archimedes spirals: Demonstrate how to draw Archimedes spiral that approximately fills ¼ of an unlined page of standard (letter) paper. The lines of the spiral should be approximately 1.3 cm (0.5 inch) apart. Then ask the subject to copy the spiral. Test and score each hand separately. Use a ballpoint pen. The pen should be held such that no part of the limb touches the table. Secure the paper on the table in a location that is suitable for the patient's style of drawing. Score the tremor in the spiral, not the movement of the limb. Each limb is assessed and scored individually. 0=normal. 1=slight: tremor barely visible. 2=mild: obvious tremor. 3=moderate: portions of figure not recognizable. 4=severe: figure not recognizable.

7) Handwriting: Have patient write the standard sentence “This is a sample of my best handwriting” using the dominant hand only. Patients must write cursively (i.e., no printing). They cannot hold or stabilize their hand with the other hand. Use a ballpoint pen. Secure the paper on the table in a location that is suitable for the patient's style of writing. Score the tremor in the writing, not the movement of the limb. 0=normal. 1=slight: untidy due to tremor that is barely visible. 2=mild: legible, but with considerable tremor. 3=moderate: some words illegible. 4=severe: completely illegible.

8) Dot approximation task: The examiner makes a dot or X and instructs the subject to hold the tip of the pen “as close as possible to the dot (or center of an X) without touching it, (ideally approximately 1 mm) for 10 seconds.” Each hand is scored separately. 0=no tremor. 1=tremor is barely visible. 1.5=tremor is visible, but less than 1 cm. 2=tremor is 1-<3 cm amplitude. 2.5=tremor is 3-<5 cm amplitude. 3=tremor is 5-<10 cm amplitude. 3.5=tremor is 10-<20 cm amplitude. 4=tremor is >20 cm amplitude.

9) Standing tremor: Subjects are standing, unaided if possible. The knees are 10-20 cm apart and are flexed 10-20°. The arms are down at the subject's side. Tremor is assessed at any point on the legs or trunk. 0=no tremor. 1=barely perceptible tremor. 2=obvious but mild tremor, does not cause instability. 3=moderate tremor, impairs stability of stance. 4=severe tremor, unable to stand without assistance.

As used herein, “The Essential Tremor Rating Assessment Scale (TETRAS) performance subscale part 4 upper limb tremor total score” refers to the “upper limb tremor” under metric or characteristic 4 of the TETRAS performance subscale set forth above. Each of the three tasks are performed on each limb to give a total of 6 tasks, wherein each task is scored 0, 1, 1.5, 2, 2.5, 3, 3.5, or 4, and each score is added to give a total score no greater than 24.

As used herein, “a reduction in tremor amplitude” or “the percentage change in tremor amplitude” is calculated based on the following formula (Elble R. J., Tremor Other Hyperkinet. Mov. 8:600 (2018)):

$$\frac{{T}_{f} - {T}_{i}}{{T}_{i}} = 1 {0}^{α ( {R}_{f} - {R}_{i} ) / N} - 1$$

wherein T_{f} is a final tremor amplitude;
   Tᵢ is an initial tremor amplitude;
   R_{f} is a final tremor rating;
   Rᵢ is an initial tremor rating;
   α is 0.05; and
   N=6, for 6 tasks in TETRAS performance subscale part 4 upper limb tremor total score.

The terms “initial” and “baseline” are used interchangeably herein.

As used herein, the term “trough concentration” and “C_{trough}” are used interchangeably to refer to the concentration reached by a drug, e.g., Compound 1, immediately before the next dose is administered.

As used herein, the term “filler” refers to an excipient that adds bulkiness to a pharmaceutical composition. Examples of fillers include without limitation lactose, sorbitol, cellulose (e.g., microcrystalline cellulose), a calcium phosphate, a starch, a sugar (e.g., mannitol, sucrose, or the like), or any combination thereof.

As used herein, the term “disintegrant” refers to an excipient that hydrates a pharmaceutical composition and aids in tablet dispersion. Examples of disintegrants include without limitation sodium croscarmellose, polyplasdone (i.e., cross-linked polyvinylpyrollidone), sodium starch glycolate, or any combination thereof.

As used herein, the term “lubricant” refers to an excipient that is added to pharmaceutical compositions that are pressed into tablets. The lubricant aids in compaction of granules into tablets and ejection of a tablet of a pharmaceutical composition from a die press. Examples of lubricants include magnesium stearate, stearic acid (stearin), hydrogenated oil, sodium stearyl fumarate, or any combination thereof.

As used herein, the term “glidant” refers to an excipient that imparts a pharmaceutical compositions with enhanced flow properties. Examples of glidants include without limitation colloidal silica, fumed silica, talc, or any combination thereof.

As used herein, the term “dose equivalent” means a bioequivalent dose. For example, the dose equivalent of a pharmaceutically acceptable salt of Compound 1 for a 50 mg dose of Compound 1 is the amount of the pharmaceutically acceptable salt (by weight) needed to provide a bioequivalent dose to the 50 mg dose of the free base of Compound 1.

### II. METHODS OF TREATING ESSENTIAL TREMOR

The present invention provides a method of treating essential tremor in a subject in need thereof, comprising administering to the subject Compound 1

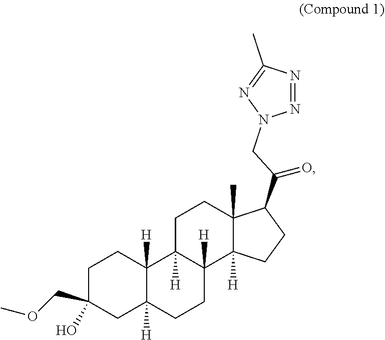

or a pharmaceutically acceptable salt thereof.

In one aspect, the present invention provides a method of treating essential tremor in a subject in need thereof, comprising administering to the subject a dose of from about 5 mg to about 80 mg amount of Compound 1

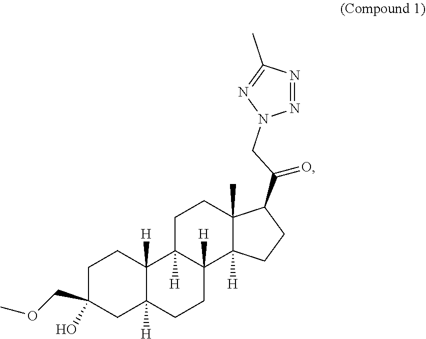

or a dose equivalent of a pharmaceutically acceptable salt thereof, at least once per day.

In some implementations, the subject is administered a dose of from about 35 mg to about 70 mg of Compound 1 or a dose equivalent of a pharmaceutically acceptable salt thereof at least once per day (e.g., QD, BID, TID, or QID). In some instances, the subject is administered a dose of from about 35 mg to about 70 mg of Compound 1 or a dose equivalent of a pharmaceutically acceptable salt thereof once per day (i.e., QD). For example, the subject is administered a dose of from about 27 mg to about 66 mg of Compound 1 or a dose equivalent of a pharmaceutically acceptable salt thereof at least once per day (e.g., QD, BID, TID, or QID). In other examples, the subject is administered a dose of from about 30 mg to about 60 mg of Compound 1 or a dose equivalent of a pharmaceutically acceptable salt thereof at least once per day (e.g., QD, BID, TID, or QID). And, in some examples, the subject is administered a dose of from about 27 mg to about 66 mg of Compound 1 or a dose equivalent of a pharmaceutically acceptable salt thereof once per day (i.e., QD). For example, the subject is administered a dose of from about 27 mg to about 66 mg of Compound 1 or a dose equivalent of a pharmaceutically acceptable salt thereof once per day, at night (e.g., at bedtime).

In some implementations, the subject is administered a dose of from about 9 mg to about 38 mg of Compound 1 or a dose equivalent of a pharmaceutically acceptable salt thereof at least once per day (e.g., QD, BID, TID, or QID). In some instances, the subject is administered a dose of from about 10 mg to about 35 mg of Compound 1 or a dose equivalent of a pharmaceutically acceptable salt thereof at least once per day (e.g., QD, BID, TID, or QID). For example, the subject is administered a dose of from about 15 mg to about 30 mg of Compound 1 or a dose equivalent of a pharmaceutically acceptable salt thereof at least once per day (e.g., QD, BID, TID, or QID). In other examples, the subject is administered a dose of from about 10 mg to about 35 mg of Compound 1 or a dose equivalent of a pharmaceutically acceptable salt thereof once per day (i.e., QD). For example, the subject is administered a dose of from about 10 mg to about 35 mg of Compound 1 or a dose equivalent of a pharmaceutically acceptable salt thereof once per day, at night (e.g., at bedtime).

In some implementations, the subject is administered a dose of from about 10 mg to about 20 mg of Compound 1 or a dose equivalent of a pharmaceutically acceptable salt thereof at least once per day (e.g., QD, BID, TID, or QID). For example, the subject is administered a dose of from about 10 mg to about 20 mg of Compound 1 or a dose equivalent of a pharmaceutically acceptable salt thereof once per day (i.e., QD). For example, the subject is administered a dose of from about 10 mg to about 20 mg of Compound 1 or a dose equivalent of a pharmaceutically acceptable salt thereof once per day, at night (e.g., at bedtime).

In some implementations, the subject is administered a dose of from about 25 mg to about 35 mg of a compound of Compound 1 or a dose equivalent of a pharmaceutically acceptable salt thereof at least once per day (e.g., QD, BID, TID, or QID). For example, the subject is administered a dose of from about 25 mg to about 35 mg of Compound 1 or a dose equivalent of a pharmaceutically acceptable salt thereof once per day (i.e., QD). In other examples, the subject is administered a dose of from about 25 mg to about 35 mg of Compound 1 or a dose equivalent of a pharmaceutically acceptable salt thereof once per day, at night (e.g., at bedtime).

In some implementations, the subject is administered a dose of from about 40 mg to about 50 mg of Compound 1 or a dose equivalent of a pharmaceutically acceptable salt thereof at least once per day (e.g., QD, BID, TID, or QID). For example, the subject is administered a dose of from about 40 mg to about 50 mg of Compound 1 or a dose equivalent of a pharmaceutically acceptable salt thereof once per day (i.e., QD). In other examples, the subject is administered a dose of from about 40 mg to about 50 mg of Compound 1 or a dose equivalent of a pharmaceutically acceptable salt thereof once per day, at night (e.g., at bedtime).

In some implementations, the subject is administered a dose of from about 55 mg to about 65 mg of Compound 1 or a dose equivalent of a pharmaceutically acceptable salt thereof at least once per day (e.g., QD, BID, TID, or QID). For example, the subject is administered a dose of from about 55 mg to about 65 mg of Compound 1 or a dose equivalent of a pharmaceutically acceptable salt thereof once per day (i.e., QD). In other examples, the subject is administered a dose of from about 55 mg to about 65 mg of Compound 1 or a dose equivalent of a pharmaceutically acceptable salt thereof once per day, at night (e.g., at bedtime).

In one aspect, the present invention provides a method of treating essential tremor in a subject in need thereof, comprising administering to the subject a therapeutically effective amount of Compound 1

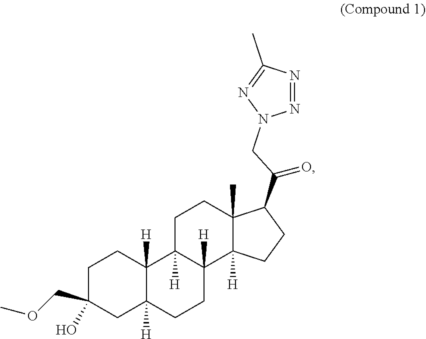

or a pharmaceutically acceptable salt thereof, wherein the subject experiences a reduction in tremor amplitude of at least about 5% following treatment onset.

In some implementations, the subject is administered a dose of from about 5 mg to about 80 mg of Compound 1 or a dose equivalent of a pharmaceutically acceptable salt thereof at least once per day.

In some implementations, the subject experiences a reduction in tremor amplitude of at least about 15% following treatment onset. For example, the subject experiences a reduction in tremor amplitude of at least about 30% (e.g., at least about 35%, at least about 40%, or at least about 50%) following treatment onset. In other examples, the subject experiences a reduction in tremor amplitude of at least about 50% following treatment onset. In some examples, the subject experiences a reduction in tremor amplitude of at least about 75% following treatment onset. And, in some examples, the subject experiences a reduction in tremor amplitude of from about 30% to about 99% (e.g., from about 30% to about 85%, from about 30% to about 80%, from about 30% to about 75%, and the like).

In some implementations, the subject experiences a reduction in tremor amplitude about 7 days following treatment onset. For example, the subject experiences a reduction in tremor amplitude about 10 days, about 12 days, about 14 days, about 16 days, about 18 days, about 20 days, about 25 days, about 30 days, or about 40 days following treatment onset.

In some implementations, the subject has an initial TETRAS performance subscale part 4 upper limb tremor total score of no greater than 12 prior to treatment onset. In some instances, the subject has an initial TETRAS performance subscale part 4 upper limb tremor total score of 1, 2, 3, 4, 5, 6, 7, 8, 9, 10, 11, or 12. In other instances, the subject has an initial TETRAS performance subscale part 4 upper limb tremor total score of from about 4 to no greater than 12. For example, the subject has an initial TETRAS performance subscale part 4 upper limb tremor total score of at least about 4 prior to treatment onset. In other examples, the subject has an initial TETRAS performance subscale part 4 upper limb tremor total score of at least about 6 prior to treatment onset. In some examples, the subject has an initial TETRAS performance subscale part 4 upper limb tremor total score of at least about 8 prior to treatment onset. In some examples, the subject has an initial TETRAS performance subscale part 4 upper limb tremor total score of at least about 10 prior to treatment onset. In other examples, the subject has an initial TETRAS performance subscale part 4 upper limb tremor total score of at least about 12 prior to treatment onset. And, in other examples, the subject has an initial TETRAS performance subscale part 4 upper limb tremor total score of at least about 13, 14, 15, 16, 17, 18, 19, 20, 21, 22, 23, or 24 prior to treatment onset.

In some implementations, the subject the subject has an initial TETRAS performance subscale part 4 upper limb tremor total score for their dominant hand of at least about 4 prior to treatment onset. In other instances, the subject the subject has an initial TETRAS performance subscale part 4 upper limb tremor total score for their dominant hand of at least about 5 prior to treatment onset. In other examples, the subject has an initial TETRAS performance subscale part 4 upper limb tremor total score for their dominant hand of at least about 5.5 prior to treatment onset. And, in other examples, the subject has an initial TETRAS performance subscale part 4 upper limb tremor total score for their dominant hand of at least about 6, 7, 8, 9, 10, 11, or 12 prior to treatment onset.

In some implementations, the subject is administered a dose of from about 5 mg to about 80 mg of Compound 1 or a dose equivalent of a pharmaceutically acceptable salt thereof. In some examples, the dose is from about 5 mg to about 60 mg. In other instances, the dose is from about 10 mg to about 60 mg. And, in some instances, the dose is about 60 mg, about 50 mg, about 45 mg, about 30 mg, about 25 mg, about 15 mg, about 10 mg, about 7.5 mg, or about 5 mg.

In some implementations, the subject is orally administered the dose (e.g., any doses described herein) of Compound 1 or a dose equivalent of a pharmaceutically acceptable salt thereof as one or more tablets, capsules, or any combination thereof. For example, the subject is orally administered the dose of Compound 1 or a dose equivalent of a pharmaceutically acceptable salt thereof as a one or two tablets.

In some implementations, Compound 1 or a dose equivalent of a pharmaceutically acceptable salt thereof is administered at least once per day. For example, Compound 1 or a dose equivalent of a pharmaceutically acceptable salt thereof is administered once or twice per day.

In some implementations, Compound 1 or a dose equivalent of a pharmaceutically acceptable salt thereof is administered once per day. For example, Compound 1 or a dose equivalent of a pharmaceutically acceptable salt thereof is administered once per day in the evening or in the morning. In some instances, Compound 1 or a dose equivalent of a pharmaceutically acceptable salt thereof is administered once per day in the evening. In other instances, the dose (e.g., any dose described herein) of Compound 1 or a dose equivalent of a pharmaceutically acceptable salt thereof is orally administered once per day, in the evening, as one or two tablets.

Another aspect of the present invention provides a method of treating essential tremor in a subject in need thereof, comprising administering to the subject a therapeutically effective amount of Compound 1

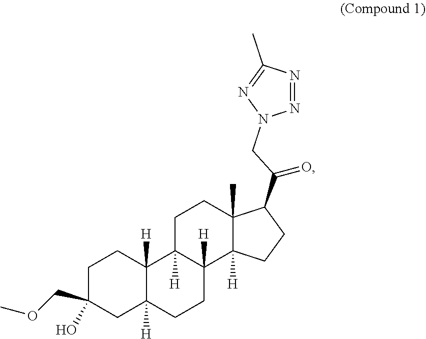

or a pharmaceutically acceptable salt thereof, wherein the subject experiences a reduction in TETRAS activities of daily living (ADL) subscale total score following treatment onset.

In some implementations, the subject experiences a reduction in TETRAS ADL subscale total score of at least about 5% following treatment onset. For example, the subject experiences a reduction in TETRAS ADL subscale total score of at least about 10% following treatment onset. In other examples, the subject experiences a reduction in TETRAS ADL subscale total score of at least about 15% following treatment onset. In other examples, the subject experiences a reduction in TETRAS ADL subscale total score of at least about 20% following treatment onset. In some examples, the subject experiences a reduction in TETRAS ADL subscale total score of from about 30% to about 50% following treatment onset.

In some implementations, the subject experiences the reduction in TETRAS ADL subscale total score about 8 days following treatment onset.

In some implementations, the subject experiences a reduction in TETRAS ADL subscale total score about 10 days following treatment onset.

In some implementations, the subject experiences a reduction in TETRAS ADL subscale total score about 15 days following treatment onset.

In some implementations, the subject experiences a reduction in TETRAS ADL subscale total score about 20 days following treatment onset.

In some implementations, the subject experiences a reduction in TETRAS ADL subscale total score about 25 days following treatment onset.

In some implementations, the subject experiences a reduction in TETRAS ADL subscale total score about 30 days following treatment onset.

In some implementations, the subject has an initial TETRAS ADL subscale total score of at least 12 prior to treatment onset.

In some implementations, the subject has an initial TETRAS ADL subscale total score of less than about 28 prior to treatment onset.

In some implementations, the subject has an initial TETRAS ADL subscale total score of at least about 20 prior to treatment onset.

In some implementations, the subject has an initial TETRAS ADL subscale total score of at least about 24 prior to treatment onset.

In some implementations, the subject has an initial TETRAS ADL subscale total score of at least about 28 prior to treatment onset.

In some implementations, the subject is administered a dose of from about 5 mg to about 80 mg of Compound 1 or a dose equivalent of a pharmaceutically acceptable salt thereof, at least once per day. For example, the subject is administered a dose of from about 10 mg to about 70 mg of Compound 1 or a dose equivalent of a pharmaceutically acceptable salt thereof, at least once per day. In other examples, the subject is administered a dose of from about 50 mg to about 70 mg of Compound 1 or a dose equivalent of a pharmaceutically acceptable salt thereof, at least once per day. In some examples, the subject is administered a dose of from about 10 mg to about 30 mg of Compound 1 or a dose equivalent of a pharmaceutically acceptable salt thereof, at least once per day. And, in some examples, the subject is administered a dose of about 10 mg, about 15 mg, about 30 mg, about 45 mg, or about 60 mg of Compound 1 or a dose equivalent of a pharmaceutically acceptable salt thereof, at least once per day.

In some implementations, the subject is administered a dose of about 60 mg of Compound 1 or a dose equivalent of a pharmaceutically acceptable salt thereof, at least once per day. For instance, the subject is administered a dose of about 60 mg of Compound 1 or a dose equivalent of a pharmaceutically acceptable salt thereof, once per day.

In some implementations, the subject is administered a dose of from about 30 mg to about 50 mg of Compound 1 or a dose equivalent of a pharmaceutically acceptable salt thereof, at least once per day.

In some implementations, the subject is administered a dose of about 45 mg of Compound 1 or a dose equivalent of a pharmaceutically acceptable salt thereof, at least once per day.

In some implementations, the subject is administered a dose of from about 20 mg to about 40 mg of Compound 1 or a dose equivalent of a pharmaceutically acceptable salt thereof, at least once per day.

In some implementations, the subject is administered a dose of from about 5 mg to about 20 mg of Compound 1 or a dose equivalent of a pharmaceutically acceptable salt thereof, at least once per day. In other implementations, the subject is administered a dose of from about 5 mg, about 7.5 mg, about 10 mg, or about 15 mg of Compound 1 or a dose equivalent of a pharmaceutically acceptable salt thereof, at least once per day.

In some implementations, Compound 1, or a pharmaceutically acceptable salt thereof, in any dose described herein, is administered at least once per day for at least about 28 days.

In some implementations, Compound 1, or a pharmaceutically acceptable salt thereof, in any dose described herein, is administered at least once per day chronically.

In some implementations, Compound 1, or the pharmaceutically acceptable salt thereof, is administered once per day.

In some implementations, Compound 1, or a pharmaceutically acceptable salt thereof, is administered once per day for about 28 days.

In some implementations, Compound 1, or a pharmaceutically acceptable salt thereof, is administered orally. For example, Compound 1, or a pharmaceutically acceptable salt thereof, is administered as a tablet or capsule.

In some implementations, the subject has a plasma concentration of Compound 1 of at least about 175 ng/mL about 8 days following treatment onset. For example, the subject has a plasma concentration of Compound 1 of about 200 ng/mL about 8 days following treatment onset. In other examples, the subject has a plasma concentration of Compound 1 of from about 200 ng/mL to about 250 ng/mL about 8 days following treatment onset.

In another example, the subject has a plasma concentration of Compound 1 at least about 210 ng/mL (e.g., at least about 250 ng/mL, at least about 275 ng/mL, at least about 300 ng/mL, at least about 330 ng/mL, or at least about 400 ng/mL) about 15 days following treatment onset. For example, the subject has a plasma concentration of Compound 1 of from about 210 ng/mL to about 475 ng/mL about 15 days following treatment onset. In another example, the subject has a plasma concentration of Compound 1 of from about 210 ng/mL to about 450 ng/mL (e.g., from about 210 ng/mL to about 350 ng/mL, from about 210 ng/mL to about 280 ng/mL, from about 320 ng/mL to about 420 ng/mL, or from about 275 ng/mL to about 490 ng/mL) about 15 days following treatment onset.

In another example, the subject has a plasma concentration of Compound 1 of at least about 230 ng/mL about 22 days following treatment onset. For example, the subject has a plasma concentration of Compound 1 of from about 230 ng/mL to about 390 ng/mL about 22 days following treatment onset.

In another example, the subject has a plasma concentration of Compound 1 of greater than about 250 ng/mL about 29 days following treatment onset. For example, the subject has a plasma concentration of Compound 1 of from about 270 ng/mL to about 450 ng/mL about 29 days following treatment onset.

Another aspect of the present invention provides a method of treating essential tremor in a subject in need thereof, comprising administering to the subject an initial dose of from about 5 mg to about 80 mg of Compound 1

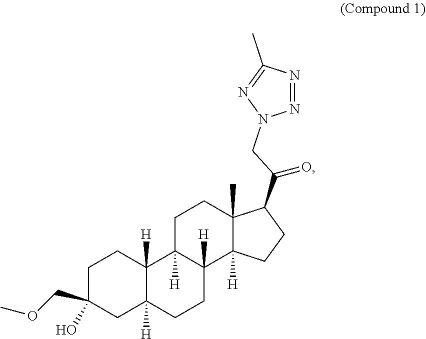

or a dose equivalent of a pharmaceutically acceptable salt thereof, wherein the initial dose is administered at least once per day.

In some implementations, the initial dose is administered from onset of treatment for a duration of from about 1 to about 30 days. For example, the initial dose is administered from onset of treatment for a duration of about 28 days. In other implementations, the initial dose is administered from onset of treatment for a duration of up to about 7 days, up to about 14 days, up to about 21 days, or up to about 28 days. In some implementations, the initial dose is administered from onset of treatment for a duration of up to about 1 month, up to about 2 months, up to about 3 months, up to about 4 months, up to about 5 months, or up to about 6 months. And, in some implementations, the initial dose is administered from onset of treatment for a duration of at least about 30 days.

In some implementations, the initial dose is about 60 mg. In some implementations, the initial dose is from about 10 mg to about 70 mg. In other implementations, the initial dose is of from about 50 mg to about 70 mg. In further implementations, the initial dose is of from about 10 mg to about 30 mg. And, in some implementations, the initial dose is about 10 mg, about 15 mg, about 30 mg, about 45 mg or about 60 mg.

In some implementations, the initial dose is of from about 30 mg to about 50 mg. In other implementations, the initial dose is about 45 mg. And, in some implementations, the initial dose is about 30 mg, about 35 mg, about 40 mg, about 45 mg, or about 50 mg.

In some implementations, the initial dose is of from about 20 mg to about 40 mg.

In some implementations, the initial dose is of from about 5 mg to about 20 mg. In other implementations, the initial dose is of about 5 mg, 7.5 mg, 10 mg, or 15 mg.

Some implementations comprise administering to the subject a second dose comprising from about 25 mg to about 50 mg of Compound 1, or a dose equivalent of a pharmaceutically acceptable salt thereof, wherein the second dose is administered on the day following the last administration of the initial dose. In some examples, the second dose comprises from about 30 mg to about 45 mg of Compound 1, or a dose equivalent of a pharmaceutically acceptable salt thereof. In other examples, the second dose comprises from about 25 mg to about 35 mg of Compound 1, or a dose equivalent of a pharmaceutically acceptable salt thereof. And, in some implementations, the second dose comprises about 5 mg, about 7.5 mg, about 10 mg, about 15 mg, about 20 mg, about 25 mg, about 30 mg, about 35 mg, about 40 mg, about 45 mg, or about 50 mg of Compound 1, or a dose equivalent of a pharmaceutically acceptable salt thereof.

In some implementations, the second dose is administered at least once per day. For example, the second dose is administered once per day.

In some implementations, the second dose is administered for a duration of about 60 days or less. In other implementations, the second dose is administered for a duration of about 50 days or less, 45 days or less, 40 days or less, 35 days or less, 30 days or less, 25 days or less, 20 days or less, 15 days or less, 10 days or less, or 5 days or less. And, in some implementations, the second dose is administered for a duration of up to about 7 days, up to about 14 days, up to about 21 days, up to about 28 days, up to about 1 month, up to about 2 months, up to about 3 months, up to about 4 months, up to about 5 months, or up to about 6 months.

Some implementations comprise administering to the subject a third dose comprising from about 5 mg to about 24 mg of Compound 1, or a dose equivalent of a pharmaceutically acceptable salt thereof, wherein the third dose is administered on the day following the last administration of the second dose. In some examples, the third dose comprises from about 5 mg to about 20 mg of Compound 1, or a dose equivalent of a pharmaceutically acceptable salt thereof. In other examples, the third dose comprises from about 7.5 mg to about 17.5 mg of Compound 1, or a dose equivalent of a pharmaceutically acceptable salt thereof. In other implementations, the third dose comprises from about 20 mg to about 40 mg of Compound 1, or a dose equivalent of a pharmaceutically acceptable salt thereof. In some implementations, the third dose comprises about 30 mg of Compound 1, or a dose equivalent of a pharmaceutically acceptable salt thereof. And, in some implementations, the third dose comprises about 5 mg, about 7.5 mg, about 10 mg, about 12.5 mg, about 15 mg, about 17.5 mg, about 20 mg, about 22.5 mg, about 25 mg, about 27.5 mg, or about 30 mg of Compound 1, or a dose equivalent of a pharmaceutically acceptable salt thereof.

In some implementations, the third dose is administered at least once per day. For example, the third dose is administered once per day.

In some implementations, the third dose is administered for a duration of about 60 days or less. In other implementations, the third dose is administered for a duration of about 50 days or less, 45 days or less, 40 days or less, 35 days or less, 30 days or less, 25 days or less, 20 days or less, 15 days or less, 10 days or less, or 5 days or less. And, in some implementations, the third dose is administered for a duration of up to about 7 days, up to about 14 days, up to about 21 days, up to about 28 days, up to about 1 month, up to about 2 months, up to about 3 months, up to about 4 months, up to about 5 months, or up to about 6 months.

In some implementations, if the subject experiences a treatment-emergent adverse event (TEAE) after administration of the initial dose, then the subject is administered a second dose (such as any second dose described herein) at least once per day (e.g., once per day) on the day following the last administration of the initial dose. In such implementations, the second dose may be administered for a duration of about 60 days or less. In other implementations, the second dose is administered for a duration of about 50 days or less, 45 days or less, 40 days or less, 35 days or less, 30 days or less, 25 days or less, 20 days or less, 15 days or less, 10 days or less, or 5 days or less. And, in some implementations, the second dose is administered for a duration of up to about 7 days, up to about 14 days, up to about 21 days, up to about 28 days, up to about 1 month, up to about 2 months, up to about 3 months, up to about 4 months, up to about 5 months, or up to about 6 months.

In some implementations, the TEAE is selected from somnolence, dizziness, fatigue, balance disorder, diplopia, dysarthria, gait disturbance, disturbance in attention, myoclonus, headache, diarrhea, paraesthesia, lethargy, mental status changes, speech disorder, coordination abnormal, asthenia, insomnia, urinary tract infection, irritability, irritability, myalgia, or any combination thereof.

In some implementations, if the TEAE remains unabated after administration of the second dose, the subject experiences another TEAE, or both, then the subject is administered a third dose (such as any third dose described herein) at least once per day (e.g., once per day) on the day following the last administration of the second dose. In such implementations, the third dose may be administered for a duration of about 60 days or less. In other implementations, the third dose is administered for a duration of about 50 days or less, 45 days or less, 40 days or less, 35 days or less, 30 days or less, 25 days or less, 20 days or less, 15 days or less, 10 days or less, or 5 days or less. And, in some implementations, the third dose is administered for a duration of up to about 7 days, up to about 14 days, up to about 21 days, up to about 28 days, up to about 1 month, up to about 2 months, up to about 3 months, up to about 4 months, up to about 5 months, or up to about 6 months.

Another aspect of the present invention provides a method of treating essential tremor in a subject in need thereof, comprising administering to the subject a therapeutically effective amount of Compound 1

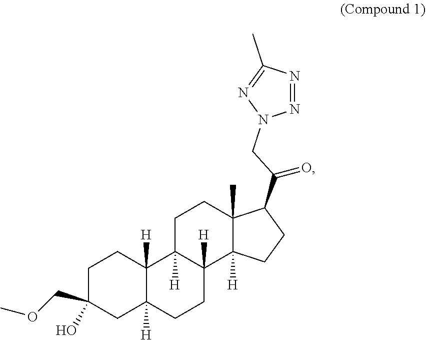

or a pharmaceutically acceptable salt thereof, wherein the subject has a plasma concentration of Compound 1 of from about 100 ng/mL to about 450 ng/mL about 8 days following treatment onset.

In some implementations, the subject has a plasma concentration of Compound 1 of from about 150 ng/mL to about 400 ng/mL about 8 days following treatment onset. For example, the subject has a plasma concentration of Compound 1 of from about 175 ng/mL to about 400 ng/mL about 8 days following treatment onset. In other examples, the subject has a plasma concentration of Compound 1 of from about 175 ng/mL to about 250 ng/mL about 8 days following treatment onset. In other examples, the subject has a plasma concentration of Compound 1 of from about 200 ng/mL to about 250 ng/mL about 8 days following treatment onset. In some examples, the plasma concentration as described herein is the plasma concentration, C_{trough}.

In some implementations, Compound 1, or the pharmaceutically acceptable salt thereof, is administered orally. For example, Compound 1, or the pharmaceutically acceptable salt thereof, is administered as a tablet or capsule.

Another aspect of the present invention provides a method of treating essential tremor in a subject in need thereof, comprising administering to the subject a therapeutically effective amount of Compound 1

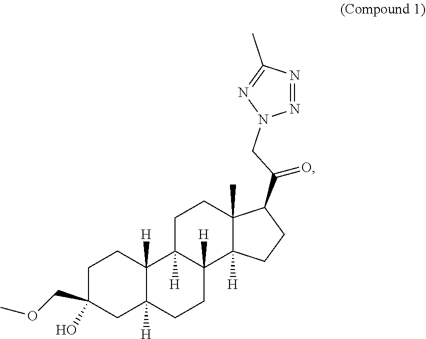

or a pharmaceutically acceptable salt thereof, wherein the subject has a plasma concentration of Compound 1 of from about 210 ng/mL to about 475 ng/mL at least about 15 days following treatment onset.

In some implementations, the subject has a plasma concentration of Compound 1 of from about 210 ng/mL to about 450 ng/mL about 15 days following treatment onset. For example, the subject has a plasma concentration of Compound 1 of from about 210 ng/mL to about 350 ng/mL about 15 days following treatment onset. For example, the subject has a plasma concentration of the compound of Compound 1 of from about 210 ng/mL to about 280 ng/mL about 15 days following treatment onset. In other examples, the subject has a plasma concentration of Compound 1 of from about 320 ng/mL to about 420 ng/mL about 15 days following treatment onset. And, in some examples, the subject has a plasma concentration of Compound 1 of from about 275 ng/mL to about 490 ng/mL about 15 days following treatment onset. In some examples, the plasma concentration as described herein is the plasma concentration, C_{trough}.

In some implementations, Compound 1, or the pharmaceutically acceptable salt thereof, is administered orally. For example, Compound 1, or the pharmaceutically acceptable salt thereof, is administered as a tablet or capsule.

Another aspect of the present invention provides a method of treating essential tremor in a subject in need thereof, comprising administering to the subject a therapeutically effective amount of Compound 1

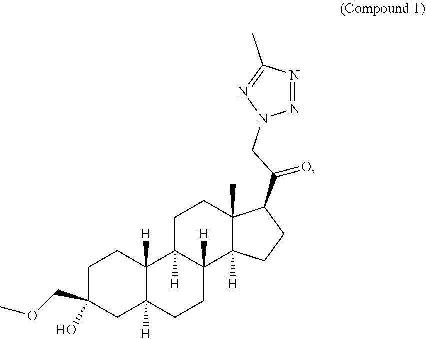

or a pharmaceutically acceptable salt thereof, wherein the subject has a plasma concentration of Compound 1 of from about 230 ng/mL to about 390 ng/mL at least about 22 days following treatment onset. In some examples, the plasma concentration as described herein is the plasma concentration, C_{trough}.

In some implementations, Compound 1, or the pharmaceutically acceptable salt thereof, is administered orally. For example, Compound 1, or the pharmaceutically acceptable salt thereof, is administered as a tablet or capsule.

### III. EXAMPLES

In order that the invention described herein may be more fully understood, the following examples are set forth. The synthetic and biological examples described in this application are offered to illustrate the compounds, pharmaceutical compositions, and methods provided herein and are not to be construed in any way as limiting their scope. In some of the tables and figures presented or referenced below, Compound 1 is referred to as “Cmpd 1.”

### Example 1: A Phase 2, Double-Blind, Placebo-Controlled, Randomized Study Evaluating the Efficacy, Safety, and Tolerability of Compound 1 in the Treatment of Individuals with Essential Tremor

### Purpose

### Primary Objective:

To assess the effect of Compound 1 compared to placebo on upper limb tremor reduction in individuals with essential tremor (ET) after 28 days of treatment.

### Secondary Objectives:

To assess the effect of Compound 1 compared to placebo on overall upper limb tremor reduction.

To assess the effect of Compound 1 compared to placebo on activities of daily living (ADLs)

To assess the effect of Compound 1 compared to placebo on overall tremor.

To evaluate the safety and tolerability of Compound 1.

### Other Objectives:

To further evaluate the safety and tolerability of Compound 1.

To evaluate the effect of Compound 1 on patient reported outcomes related to ET, sleep, depression, anxiety, quality of life, embarrassment, perceived health status, and most bothersome symptoms.

To evaluate the effect of Compound 1 on clinician reported outcomes.

To determine plasma concentrations and pharmacokinetic (PK) profile of Compound 1 following administration of Compound 1 Oral Tablets to individuals with ET.

### Study Endpoints

### Primary:

Change from baseline compared to placebo in The Essential Tremor Rating Assessment (TETRAS) performance subscale part 4 upper limb tremor score on Day 29.

### Secondary:

Change from baseline compared to placebo in TETRAS performance subscale part 4 upper limb tremor score at all timepoints other than Day 29.

Change from baseline compared to placebo in Kinesia ONE accelerometer scores.

Change from baseline compared to placebo in the following: TETRAS Scale ADL score or TETRAS Total Performance Score.

### Safety:

Incidence of treatment-emergent adverse events (TEAEs).
Safety and tolerability of Compound 1 as assessed by change from baseline in vital signs, electrocardiograms (ECGs), clinical laboratory parameters, and Columbia-Suicide Severity Rating Scale (C-SSRS).
Physician Withdrawal Checklist (PWC-20).

### Study Population

Participants were 18 to 80 years of age, inclusive, diagnosed of ET of at least 3 years duration with scores of at least 1.5 for each of the six items that comprised the combined total upper limb TETRAS (total performance subscale part 4) with the total score for the dominant upper limb (the sum of the 3 items for either the right or left upper limb, whichever is dominant) being at least 5.5, at both Screening and predose on Day 1.

### Treatment Groups

Groups received either Compound 1 as orally administered tablet(s) (each tablet providing 15 mg of Compound 1) or matching placebo at 1:1 ratio. Tablets were formulated as immediate release, white to off-white, round, film-coated tablets containing 15 mg of Compound 1, filler, disintegrant, lubricant, and glidant, wherein the tablets were formed using direct compression and coated with Opadry® II white as the film coating.

### Study Design

This is a randomized, double-blind, placebo-controlled, parallel group study to evaluate the efficacy, safety, and tolerability of Compound 1 in individuals with ET.

Participants were randomized to receive Compound 1 60 mg or placebo once daily in the morning on a 1:1 basis. If participants reported a TEAE considered by investigator to be related to Compound 1 and not tolerable, investigator might reduce the dose of Compound 1 from 60 mg in 15 mg decrements (i.e., 60 mg, to 45 mg, to 30 mg), as medically appropriate. If intolerable TEAEs persist at the 30 mg dose, Compound 1 was permanently stopped.

This study included a Screening Period of up to 28 days, a 29-day treatment period consisting of 28 days of dosing with the end of treatment visit intended to be on Day 29 at trough, and a 14-day follow-up period relative to final dose. Study patients were not taking other medications for essential tremor during the 28-day treatment period.

Efficacy and safety assessments were performed periodically during the study, and blood samples was collected for analysis of Compound 1 as outlined in the Schedule of Events. Blood samples were collected, and outcome measures were be obtained at pre-specified times over the 28-day Treatment Period.

Participants were monitored for safety during the Treatment and Follow-up Periods (through Study Day 45 [±3 days]) including monitoring for AEs/serious AEs (SAEs), routine clinical laboratory assessments, physical examination, vital signs, and ECG, etc.

### Determination of Sample Size

Assuming a 2-sided test at an alpha level of 0.05, a sample size of approximately 25 evaluable participants per treatment group would provide 85% power to detect a placebo-adjusted treatment difference of 3 points in the primary endpoint, change from baseline in TETRAS performance subscale part 4 upper limb tremor score at Day 29 assuming standard deviation (SD) of 3 points.

Assuming a non-evaluability rate of 15% and 1:1 randomization, approximately 60 participants would be randomized. Additional participants may be enrolled if the dropout rate is greater than 15%.

### Analysis Sets

All Participants: included all participants who have given written informed consent. This analysis set was used for subject disposition.

Full Analysis Set: included all randomized participants who received any amount of Compound 1 and have a baseline and at least one postbaseline efficacy assessment. The Efficacy Set was used to analyze the efficacy data. For the Full Analysis Set, N=67.

Safety Set: defined as all participants who received at least one dose of Compound 1. The Safety Set was used to provide descriptive summaries of the safety data.

### Statistical Analysis

### Efficacy

The change from baseline HAM-D total score was analyzed using a mixed effects model for repeated measures (MMRM).

The model included the change from baseline at each visit as the dependent variable, treatment, baseline TETRAS performance subscale part 4 upper limb tremor score, assessment timepoint, and timepoint-by-treatment as explanatory variables. All explanatory variables were treated as a fixed effect. The main comparison was (difference in least mean square [LSMEAN]) between Compound 1 and placebo at the 29-day timepoint.

Model-based point estimates (i.e., LSMEAN, 95% confidence intervals, and p-values) were reported where applicable. An unstructured covariance structure was used to model the within-subject errors. If there was a convergence issue with the unstructured covariance model, Toeplitz, compound symmetry or Autoregressive (1) (AR[1]) covariance structure was used, following this sequence until convergence was achieved. If the model still didn't converge with AR(1) structure, no results would be reported. When the covariance structure is not UN, the sandwich estimator for the variance-covariance matrix would be derived, using the EMPIRICAL option in the PROC MIXED statement in SAS.

Similarly, an MMRM was be used for the analysis of the following variables: changes from baseline in TETRAS performance subscale part 4, 6, 7, and 8 combined score, and TETRAS ADL score. For each model, the comparison of interest was between Compound 1 tablets and matching placebo at the 15-day time point. Model-based point estimates (i.e., LS means), 95% confidence intervals, and p-values were reported.

The percentage change in tremor amplitude is calculated based on the following formula:

$$\frac{{T}_{f} - {T}_{i}}{{T}_{i}} = 1 {0}^{α ( {R}_{f} - {R}_{i} ) / N} - 1$$

wherein T_{f} is a final tremor amplitude;
Tᵢ is an initial tremor amplitude;
R_{f} is a final tremor rating;
Rᵢ is an initial tremor rating;
α is 0.05; and
N=6, for 6 items in TETRAS performance subscale part 4 upper limb tremor total score.

### Subgroup Analysis for Primary Endpoint

The MMRM analysis for change from baseline in the HAM-D total score analysis was repeated for the following subgroups:
Age group: <65, 65-80 years
Sex: Male, Female
Race: White, Black or African American, Other
BMI (≤18.5, 18.5-<25, 25-<30, ≥30 kg/m²)
Age at essential tremor diagnoses (<40 years, 40-60, >60 years)
Age participant thinks essential tremor started (<40 years, 40-60, >60 years)
Years with essential tremor diagnosis (3-<6 years, 6-10 years, >10 years)
Years since participant thinks essential tremor started (3-<6 years, 6-10 years, >10 years)
Alcohol helps tremor: Yes (Intermediate, Worked Well), No (No, a little), NA
Baseline TETRAS performance subscale part 4 upper limb tremor score (<12, ≥12)

Observed values and change/percent change from baseline to the Day 29 morning assessment (Day 29—Baseline) (and all other time points) in TETRAS Subscale Part 4 score were summarized by treatment group and subgroup.

In addition, the LS mean change from baseline (and 95% CI of the mean) in TETRAS Subscale Part 4 score at Day 29 based on MMRM results were plotted by treatment group and subgroup.

### Safety

The safety and tolerability of Compound 1 were evaluated by adverse events, concomitant medication usage, changes from baseline in physical examination, vital signs, clinical laboratory evaluations, and 12-lead ECG. Suicidality was monitored by the C-SSRS. Safety data were listed by subject and by study drug and summarized descriptively. All safety summaries were performed on the Safety Set.

The incidence of adverse events were summarized by system organ class, and preferred term for subjects in Safety Set.

### Results

### Subject Disposition

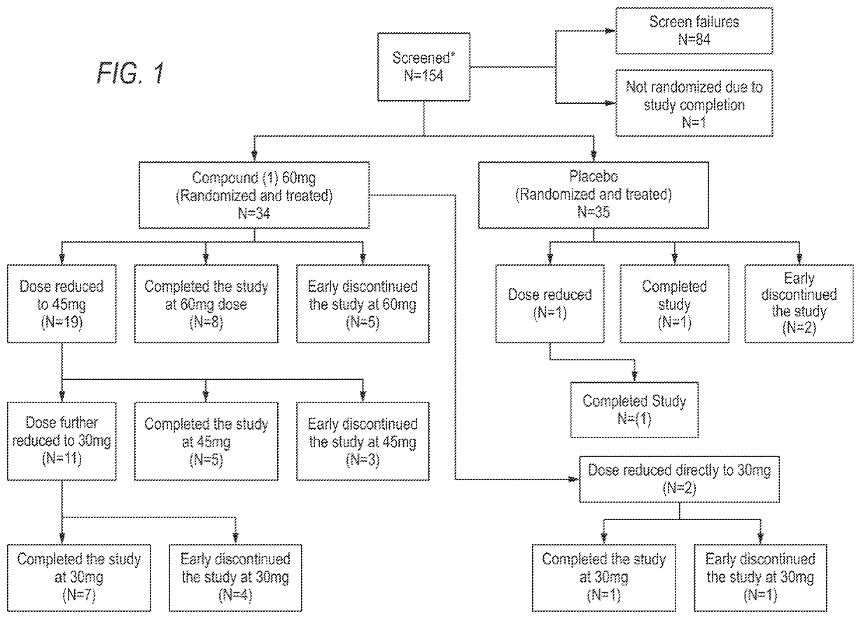

The subject disposition is summarized in FIG. 1. A total of 154 unique participants were screened, of which 84 participants were screen failures and one participant did not randomize due to study completion. 69 participants were randomized in a 1:1 ratio to receive Compound 1 60 mg (34 participants) or placebo (35 participants) and everybody received study drug.

### Demographics and Baseline Characteristics

The demographic and baseline characteristics of the participants entering the study were well balanced between the treatment groups, except for gender.

TABLE 1
Demographics and baseline characteristics.

| | Placebo | | Compound 1 | | Overall | |
|---|---|---|---|---|---|---|
| Parameter | N = 35 | | N = 34 | | N = 69 | |
| Age (Years) | | | | | | |
| n | 35 | | 34 | | 69 | |
| Mean (SD) | 64.7 | (13.18) | 69.4 | (6.91) | 67.0 | (10.75) |
| Median | 68.0 | | 69.0 | | 68.0 | |
| Min, Max | 24, 79 | | 55, 80 | | 24, 80 | |
| Sex, n (%) | | | | | | |
| Male | 15 | (42.9) | 22 | (64.7) | 37 | (53.6) |
| Female | 20 | (57.1) | 12 | (35.3) | 32 | (46.4) |
| Ethnicity, n (%) | | | | | | |
| Hispanic or Latino | 4 | (11.4) | 3 | (8.8) | 7 | (10.1) |
| Not Hispanic or | 29 | (82.9) | 31 | (91.2) | 60 | (87.0) |
| Latino | | | | | | |
| Not Reported | 2 | (5.7) | 0 | | 2 | (2.9) |
| Race, n (%) | | | | | | |
| American Indian or | 0 | | 0 | | 0 | |
| Alaska Native | | | | | | |
| Asian | 1 | (2.9) | 0 | | 1 | (1.4) |
| Black or African | 1 | (2.9) | 2 | (5.9) | 3 | (4.3) |
| American | | | | | | |
| Native Hawaiian or | 1 | (2.9) | 0 | | 1 | (1.4) |
| Other Pacific Islander | | | | | | |
| White | 32 | (91.4) | 32 | (94.1) | 64 | (92.8) |
| Height (cm) | | | | | | |
| n | 35 | | 34 | | 69 | |
| Mean (SD) | 171.28 | (11.078) | 172.72 | (10.282) | 171.99 | (10.639) |
| Median | 170.20 | | 174.00 | | 172.70 | |
| Min, Max | 147.0, 198.1 | | 152.0, 191.0 | | 147.0, 198.1 | |
| Weight (kg) | | | | | | |
| n | 35 | | 34 | | 69 | |
| Mean (SD) | 83.50 | (18.534) | 92.68 | (24.670) | 88.02 | (22.102) |
| Median | 81.80 | | 86.90 | | 86.00 | |
| Min, Max | 46.5, 116.0 | | 57.0, 184.3 | | 46.5, 184.3 | |
| BMI (kg/m²) | | | | | | |
| n | 35 | | 34 | | 69 | |
| Mean (SD) | 28.268 | (4.9852) | 30.991 | (7.4934) | 29.610 | (6.4465) |
| Median | 27.717 | | 29.096 | | 28.508 | |
| Min, Max | 21.52, 42.34 | | 23.53, 63.03 | | 21.52, 63.03 | |
| Dominant Hand, n (%) | | | | | | |
| Left | 4 | (11.4) | 5 | (14.7) | 9 | (13.0) |
| Right | 31 | (88.6) | 29 | (85.3) | 60 | (87.0) |

### Study Drug Exposure and Compliance

Referring to FIG. 1, in the Compound 1 group, 8/34 (23.5%) participants completed the study at 60 mg dose, 19/34 (55.9%) had dose reduced to 45 mg, 5/64 (14.7%) had early discontinuation of the study at 60 mg, and 2/34 (5.9%) had dose reduced to 30 mg from 60 mg directly. For those participants who had dose reduced to 45 mg, 5 completed the study at 45 mg, 11 had dose further reduced to 30 mg, and 3 had early discontinuation of the study at 45 mg. For those 11 participants who had dose reduced to 30 mg from 45 mg, 7 completed the study at 30 mg, and 4 had early discontinuation of the study. For those 2 participants who had dose reduced to 30 mg from 60 mg directly, 1 completed the study at 30 mg and 1 had early discontinuation of the study at 30 mg.

In the placebo group, 32/35 completed the study at initial dose, 1/35 had dose reduced and completed the study, and 2/35 early discontinued the study.

TABLE 2
Reasons for early treatment discontinuation.

| | Placebo | Cmpd 1 |
|---|---|---|
| Reason | (N = 35) | (N = 34) |
| Adverse Event | 0 | 9 (26.5%) |
| Physician Decision | 0 | 1 (2.9%) |
| Subject was lost to follow up | 1 (2.9%) | 0 |
| Withdrawal by Subject | 1 (2.9%) | 3 (8.8%) |

Mean exposure was 21.3 days (range 3-29 days) for Compound 1 and 27.2 days (range 8-31 days) for placebo.

The adherence was 94.3% with a range of (74.47%-103.57%) for Compound 1 and 100.2% (2.73) with range of (96.43%-110.71%) for placebo.

Tremor Amplitude (Change from Baseline in TETRAS Performance Subscale Part 4 Upper Limb Tremor Score at Day 29):

### Full Analysis Set:

### Baseline Mean (SD):

Placebo: 12.28 (1.698)
Compound 1: 12.82 (1.727)
   Change from Baseline at Day 29:

### LSMean(SE):

Placebo: −1.24 (0.349)
Compound 1: −2.31 (0.401)

### Treatment Difference:

Compound 1 vs. Placebo
−1.07 (−2.14, −0.00)
P-value=0.0491

TABLE 3
Model-based estimates of treatment (LS) mean difference
in change from baseline in TETRAS performance subscale
part 4 upper limb tremor total score (Full Analysis Set).

| | Model-based LSMean (SE) | | | | |
|---|---|---|---|---|---|
| Visit | Placebo | Cmpd 1 | Difference (95% CI) | | P-value |
| Day 8 | −0.86 (0.273) | −1.67 (0.287) | −0.81 | (−1.60, −0.01) | 0.0468 |
| Day 15, predose | −1.31 (0.312) | −1.78 (0.344) | −0.48 | (−1.41, 0.46) | 0.3124 |
| Day 15, 5 hr postdose | −2.24 (0.400) | −2.64 (0.452) | −0.40 | (−1.61, 0.81) | 0.5148 |
| Day 15, 8 hr postdose | −2.09 (0.389) | −2.36 (0.441) | −0.27 | (−1.45, 0.91) | 0.6452 |
| Day 22 | −1.33 (0.318) | −2.10 (0.360) | −0.77 | (−1.73, 0.20) | 0.1171 |
| Day 29 | −1.24 (0.349) | −2.31 (0.401) | −1.07 | (−2.14, −0.00) | 0.0491 |
| Day 42 | −1.85 (0.378) | −1.23 (0.407) | 0.62 | (−0.50, 1.73) | 0.2724 |

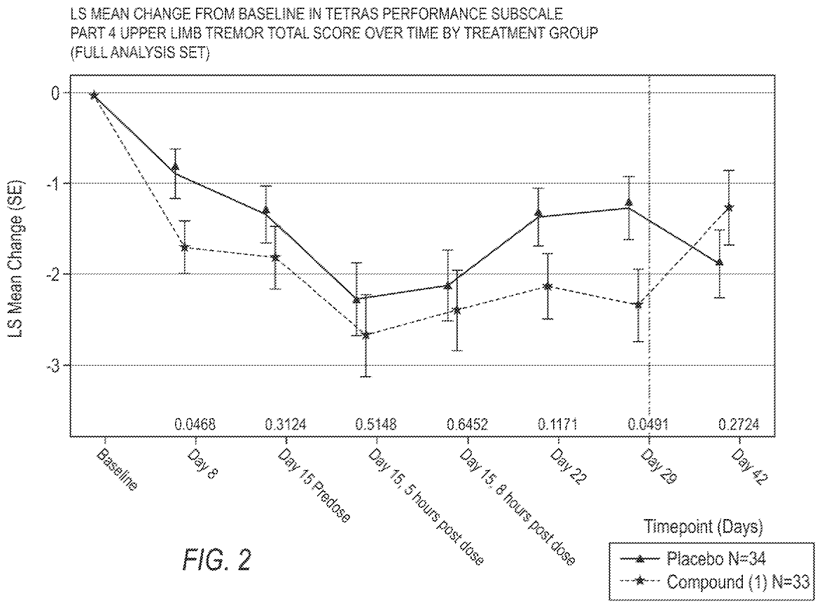

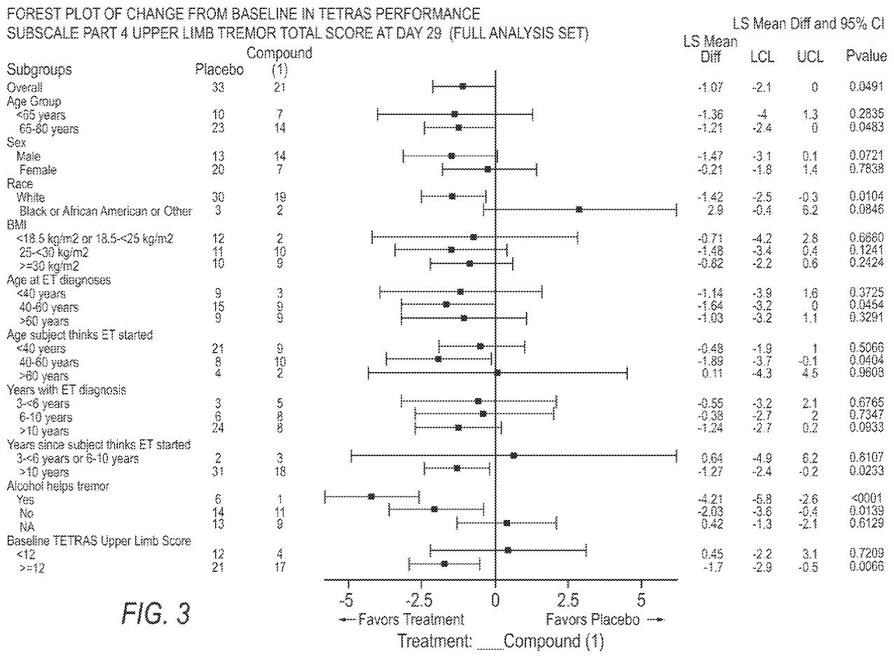

For the Full Analysis Set, LS Mean change from baseline in TETRAS performance subscale part 4 upper limb tremor total score over time by treatment group is shown in FIG. 2. A forest plot of change from baseline in TETRAS performance subscale part 4 upper limb total score at day 29 is shown in FIG. 3.

TABLE 4
Summary of change from baseline in TETRAS performance subscale part
4 upper limb tremor total score and by TETRAS severity group.

| | | | Baseline TETRAS Upper | | Baseline TETRAS Upper | |
|---|---|---|---|---|---|---|
| | Full Analysis Set | | Limb Part 4 < 12 | | Limb Part 4 ≥ 12 | |
| | Placebo | Cmpd 1 | Placebo | Cmpd 1 | Placebo | Cmpd 1 |
| | (N = 34) | (N = 33) | (N = 12) | (N = 8) | (N = 22) | (N = 25) |
| N at Day 29 | 33 | 21 | 12 | 4 | 21 | 17 |
| LSMean (SE) CFB | −1.24 | −2.31 | −1.50 | −1.06 | −1.05 | −2.75 |
| | (0.349) | (0.401) | (0.667) | (1.036) | (0.412) | (0.426) |
| LSMean Difference | | −1.07 | | 0.45 | | −1.70 |
| (CI) | | (−2.14, −0.00) | | (−2.18, 3.07) | | (−2.90, −0.50) |
| P-value | | 0.0491 | | 0.7209 | | 0.0066 |
| Mean (SD) % CFB | −9.68 | −18.05 | −13.56 | −7.76 | −7.46 | −20.47 |
| | (15.659) | (17.611) | (21.901) | (12.397) | (10.692) | (18.061) |
| % CFB in Tremor | −21.17 | −35.80 | −25.01 | −18.40 | −18.24 | −41.00 |
| Amplitude | | | | | | |

Percent reduction from baseline in tremor amplitude: 21 for placebo versus 36 for Compound 1 for Full Analysis Set.

Percent reduction from baseline in tremor amplitude: 18% for placebo versus 41% for Compound 1 for subjects with baseline TETRAS performance subscale part 4 upper limb tremor total score ≥12).

TABLE 6
Frequency distribution of percent change from baseline in tremor amplitude.

| % CFB in | | | | | Baseline TETRAS Upper | | | | Baseline TETRAS Upper | | | |
|---|---|---|---|---|---|---|---|---|---|---|---|---|
| Tremor | Full Analysis Set | | | | Limb Part 4 < 12 | | | | Limb Part 4 ≥ 12 | | | |
| Amplitude | Placebo | | Cmpd 1 | | Placebo | | Placebo | | Cmpd 1 | | Placebo | |
| Categories | (N = 34) | | (N = 33) | | (N = 12) | | (N = 8) | | (N = 22) | | (N = 25) | |
| n at Day 29 | 33 | | 21 | | 12 | | 4 | | 21 | | 17 | |
| >75% | 0 | | 2 | (9.5) | 0 | | 0 | | 0 | | 2 | (11.8) |
| 50-<75% | 5 | (15.2) | 3 | (14.3) | 3 | (25) | 0 | | 2 | (9.5) | 3 | (17.6) |
| 30-<50% | 4 | (12.1) | 6 | (28.6) | 2 | (16.7) | 1 | (25) | 2 | (9.5) | 5 | (29.4) |
| 15-<30% | 7 | (21.2) | 5 | (23.8) | 1 | (8.3) | 1 | (25) | 6 | (28.6) | 4 | (23.5) |
| <15% | 17 | (51.5) | 5 | (23.8) | 6 | (50) | 2 | (50) | 11 | (52.4) | 3 | (17.6) |

### Subgroup of Full Analysis Set, Baseline TETRAS Performance Part 4 Upper Limb Score ≥12:

TABLE 6
Model-based estimates of treatment (LS) mean difference in change from baseline
in TETRAS performance subscale part 4 upper limb tremor total score (Baseline
TETRAS performance subscale part 4 upper limb total score ≥ 12).

| | Model-based LSMean (SE) | | | | |
|---|---|---|---|---|---|
| Visit | Placebo | Cmpd 1 | Difference (95% CI) | | P-value |
| Day 8 | −0.66 (0.352) | −1.99 (0.340) | −1.33 | (−2.32, −0.34) | 0.0096 |
| Day 15, predose | −0.78 (0.368) | −2.15 (0.371) | −1.37 | (−2.43, −0.32) | 0.0121 |
| Day 15, 5 hr postdose | −1.52 (0.442) | −3.12 (0.449) | −1.60 | (−2.88, −0.33) | 0.0147 |
| Day 15, 8 hr postdose | −1.46 (0.437) | −2.84 (0.445) | −1.38 | (−2.65, −0.12) | 0.0323 |
| Day 22 | −1.08 (0.376) | −2.56 (0.383) | −1.48 | (−2.56, −0.39) | 0.0088 |
| Day 29 | −1.05 (0.412) | −2.75 (0.426) | −1.70 | (−2.90, −0.50) | 0.0066 |
| Day 42 | −1.80 (0.479) | −1.59 (0.475) | 0.22 | (−1.14, 1.58) | 0.7486 |

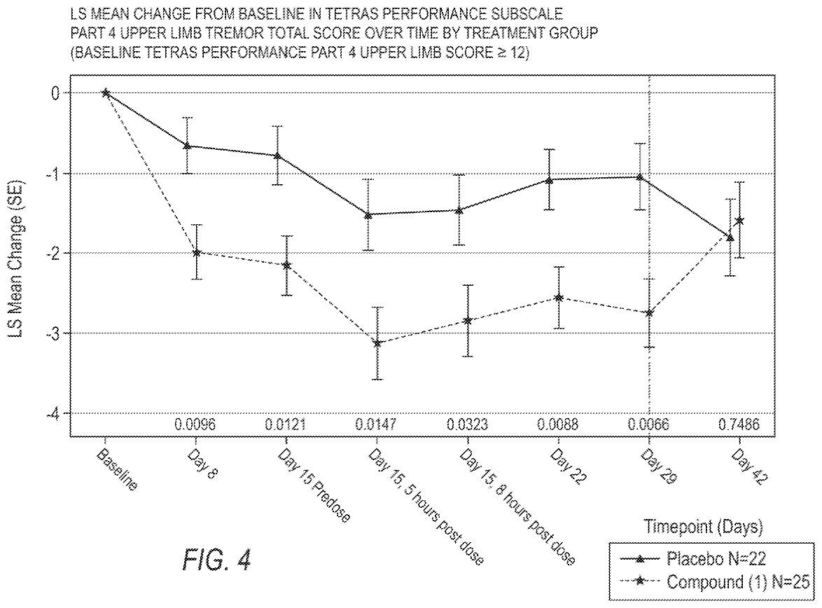

For those patients having a baseline TETRAS performance subscale part 4 upper limb total score ≥12, LS Mean change from baseline in TETRAS performance subscale part 4 upper limb tremor total score over time by treatment group is shown in FIG. 4.

Per-Protocol Set: Per-Protocol Set excludes 9 patients who had major protocol deviations: 7 in Compound 1 and 2 in placebo; 5 with positive cotinine test (1 also had ET less than 3 years, and 1 had drug adherence <75), 1 had ET less than 3 years, 1 with positive THC test, 1 did not have dominant hand TETRAS Part 4 upper limb score 5.5, 1 took Compound 1 prior to the Day 29 TETRAS assessment).

TABLE 7
Model-based estimates of treatment (LS) mean difference
in change from baseline in TETRAS performance subscale
part 4 upper limb tremor total score (Per-Protocol Set).

| | Model-based LSMean (SE) | | | | |
|---|---|---|---|---|---|
| Visit | Placebo | Cmpd 1 | Difference (95% CI) | | P-value |
| Day 8 | −0.81 (0.280) | −1.78 (0.319) | −0.97 | (−1.83, −0.11) | 0.0270 |
| Day 15, predose | −1.11 (0.302) | −1.94 (0.367) | −0.83 | (−1.79, 0.13) | 0.0876 |
| Day 15, 5 hr postdose | −2.13 (0.401) | −2.83 (0.500) | −0.71 | (−1.99, 0.58) | 0.2771 |
| Day 15, 8 hr postdose | −1.96 (0.399) | −2.66 (0.500) | −0.70 | (−1.99, 0.59) | 0.2793 |
| Day 22 | −1.23 (0.329) | −2.38 (0.408) | −1.15 | (−2.20, −0.09) | 0.0332 |
| Day 29 | −1.13 (0.354) | −2.50 (0.452) | −1.37 | (−2.52, −0.21) | 0.0214 |
| Day 42 | −1.84 (0.404) | −1.48 (0.474) | 0.36 | (−0.89, 1.62) | 0.5654 |

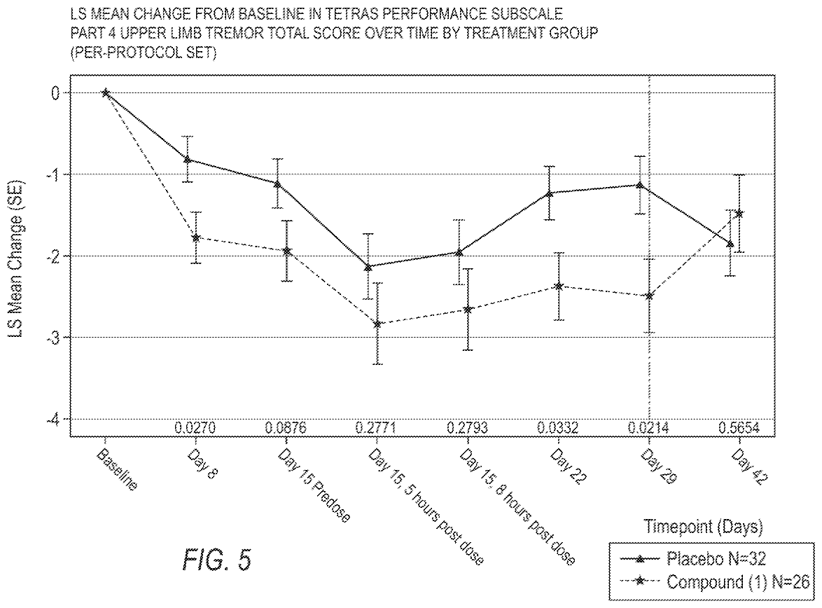

For those patients in the Per-Protocol Set, LS Mean change from baseline in TETRAS performance subscale part 4 upper limb tremor total score over time by treatment group is shown in FIG. 5.

TETRAS ADL Total Score (Change from Baseline in TETRAS ADL Total Score at Day 29):

### Full Analysis Set:

### Baseline Mean (SD):

Placebo: 26.7 (6.84)
Compound 1: 26.3 (8.50)
   Change from Baseline at Day 29:

### LSMean(SE):

Placebo: −2.87 (0.780)
Compound 1: −4.24 (0.940)

### Treatment Difference:

Compound 1 vs. Placebo
−1.37 (−3.81, 1.08)
P-value=0.2682

TABLE 8
Summary of Change from Baseline in TETRAS ADL
Total Score and By TETRAS ADL Severity Group.

| | | | Baseline TETRAS Upper | | Baseline TETRAS Upper | |
|---|---|---|---|---|---|---|
| | Full Analysis Set | | ADL < 28 | | ADL ≥ 28 | |
| | Placebo | Cmpd 1 | Placebo | Cmpd 1 | Placebo | Cmpd 1 |
| | (N = 34) | (N = 33) | (N = 18) | (N = 15) | (N = 16) | (N = 18) |
| Nat Day 29 | 33 | 21 | 18 | 10 | 15 | 11 |
| LSMean (SE) | −2.87 | −4.24 | −1.67 | −2.84 | −4.21 | −5.67 |
| CFB | (0.780) | (0.940) | (1.089) | (1.437) | (1.272) | (1.402) |
| Mean (SD) % | −10.0 | −17.4 | −8.46 | −16.52 | −11.89 | −18.10 |
| CFB | (20.67) | (14.76) | (26.090) | (14.079) | (11.954) | (16.006) |
| LSMean | | −1.37 | | −1.17 | | −1.47 |
| Difference (CI) | | (−3.81, 1.08) | | (−4.89, 2.55) | | (−5.40, 2.47) |
| P-value | | 0.2682 | | 0.5244 | | 0.4476 |

For change from baseline in TETRAS ADL subscale total score at Day 29 for the Full Analysis Set, Compound 1 was numerically superior to placebo but failed to reach statistical significance.

TABLE 9
Model-based estimates of treatment (LS) mean difference in change
from baseline in TETRAS ADL total score (Full Analysis Set).

| | Model-based LSMean (SE) | | | | |
|---|---|---|---|---|---|
| Visit | Placebo | Cmpd 1 | Difference (95% CI) | | P-value |
| Day 8 | −2.54 (0.805) | −5.34 (0.845) | −2.80 | (−5.14, −0.47) | 0.0193 |
| Day 15 | −2.47 (0.859) | −5.43 (0.945) | −2.95 | (−5.51, −0.40) | 0.0243 |
| Day 22 | −2.68 (0.802) | −5.24 (0.904) | −2.56 | (−4.98, −0.14) | 0.0385 |
| Day 29 | −2.87 (0.780) | −4.24 (0.940) | −1.37 | (−3.81, 1.08) | 0.2682 |
| Day 42 | −3.27 (0.837) | −2.14 (0.905) | 1.13 | (−1.34, 3.59) | 0.3649 |

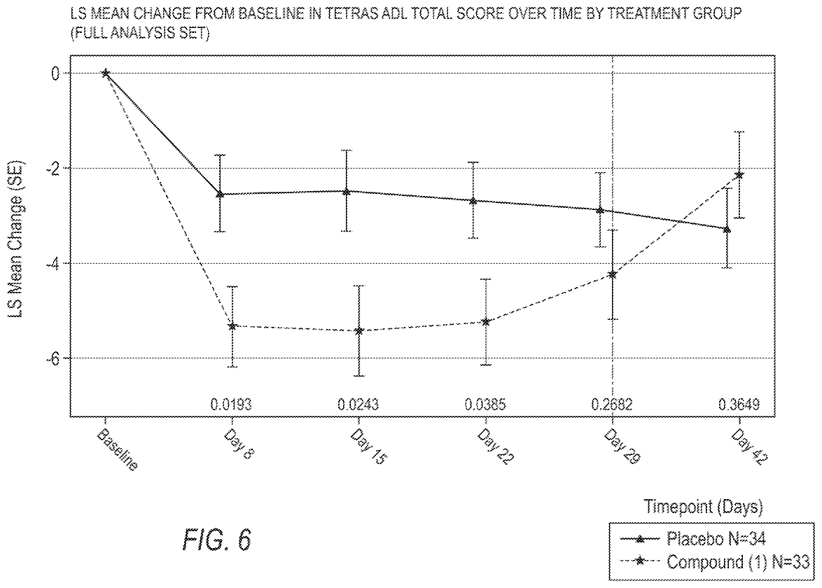

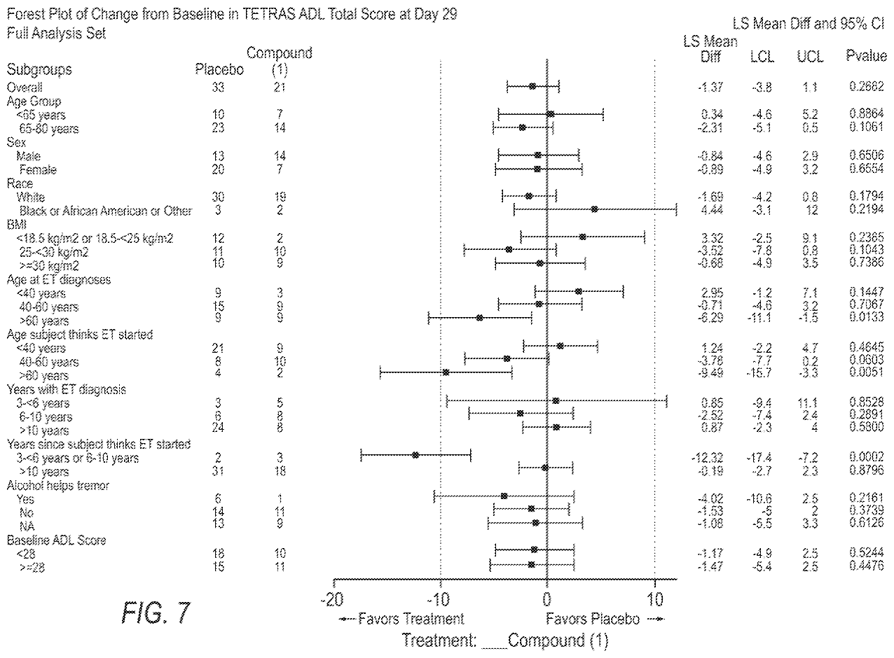

For the Full Analysis Set, LS Mean change from baseline in TETRAS ADL total score over time by treatment group is shown in FIG. 6. A forest plot of change from baseline in TETRAS ADL total score at day 29 is shown in FIG. 7.

### TETRAS Performance Subscale Total Score (Change in TETRAS Performance Subscale Total Score at Day 29):

### Full Analysis Set:

### Baseline Mean (SD):

Placebo: 27.09 (5.157)
Compound 1: 27.68 (4.618)
   Change from Baseline at Day 29:

### LSMean(SE):

Placebo: −2.90 (0.817)
Compound 1: −3.22 (0.922)

### Treatment Difference:

Compound 1 vs. Placebo
−0.32 (−2.78, 2.14)
P-value=0.7960

TABLE 10
Model-based estimates of treatment (LS) mean difference in change from baseline
in TETRAS performance subscale total score (Full Analysis Set).

| | Model-based LSMean (SE) | | | | |
|---|---|---|---|---|---|
| Visit | Placebo | Cmpd 1 | Difference (95% CI) | | P-value |
| Day 8 | −2.30 (0.743) | −3.55 (0.780) | −1.26 | (−3.41, 0.90) | 0.2478 |
| Day 15, predose | −2.55 (0.805) | −3.46 (0.878) | −0.91 | (−3.29, 1.47) | 0.4486 |
| Day 15, 5 hr postdose | −4.38 (0.892) | −5.90 (1.015) | −1.52 | (−4.22, 1.19) | 0.2662 |
| Day 15, 8 hr postdose | −4.61 (0.855) | −4.61 (0.954) | 0.01 | (−2.56, 2.57) | 0.9965 |
| Day 22 | −2.92 (0.811) | −3.16 (0.914) | −0.24 | (−2.68, 2.20) | 0.8437 |
| Day 29 | −2.90 (0.817) | −3.22 (0.922) | −0.32 | (−2.78, 2.14) | 0.7960 |
| Day 42 | −3.87 (0.761) | −1.59 (0.820) | 2.28 | (0.05, 4.52) | 0.0456 |

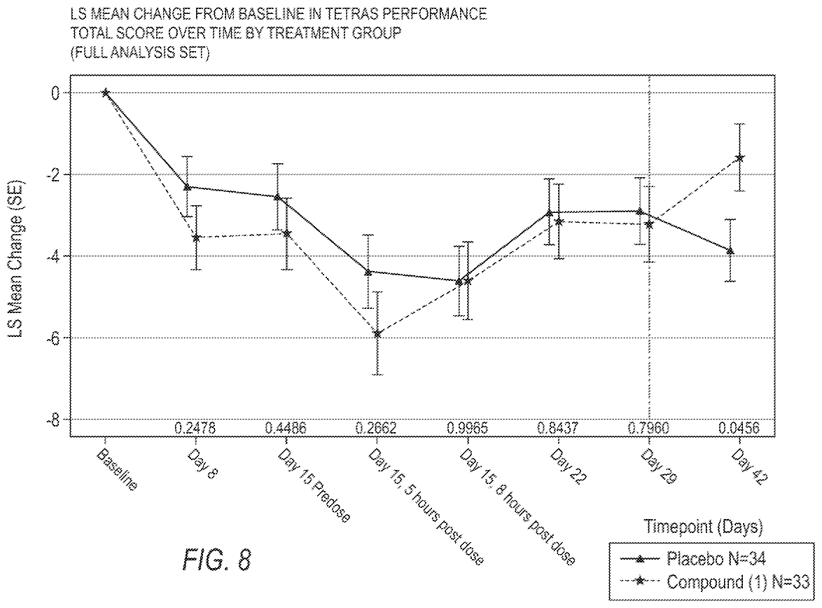

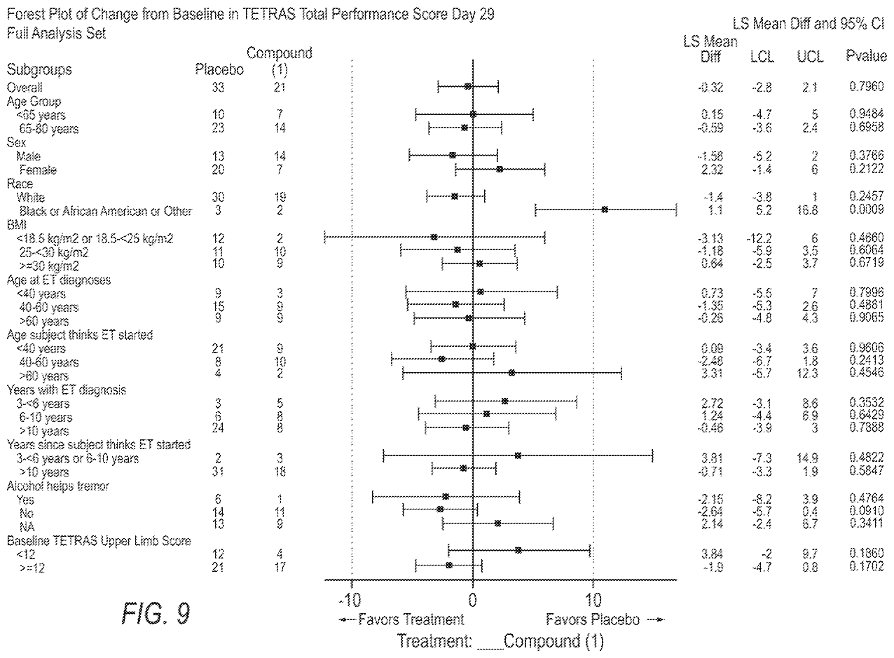

For the Full Analysis Set, LS Mean change from baseline in TETRAS performance subscale total score over time by treatment group is shown in FIG. 8. A forest plot of change from baseline in TETRAS performance subscale total score at day 29 is shown in FIG. 9.

### TETRAS Performance Subscale Part 4, 6, 7, and 8 Score (Change in TETRAS Performance Subscale Part 4, 6, 7 and 8 Score at Day 29):

### Full Analysis Set:

### Baseline Mean (SD):

Placebo: 22.82 (3.520)
Compound 1: 24.05 (3.928)
   Change from Baseline at Day 29:

### LSMean(SE):

Placebo: −2.22 (0.615)
Compound 1: −2.81 (0.699)

### Treatment Difference:

Compound 1 vs. Placebo
−0.59 (−2.45, 1.28)
P-value=0.5331

TABLE 11
Model-based estimates of treatment (LS) mean difference in change from baseline
in TETRAS performance subscale part 4/6/7/8 total score (Full Analysis Set).

| | Model-based LSMean (SE) | | | | |
|---|---|---|---|---|---|
| Visit | Placebo | Cmpd 1 | Difference (95% CI) | | P-value |
| Day 8 | −1.62 (0.550) | −2.84 (0.579) | −1.22 | (−2.82, 0.38) | 0.1337 |
| Day 15, predose | −2.08 (0.611) | −2.63 (0.671) | −0.55 | (−2.37, 1.28) | 0.5511 |
| Day 15, 5 hr postdose | −3.56 (0.740) | −4.59 (0.840) | −1.03 | (−3.28, 1.21) | 0.3608 |
| Day 15, 8 hr postdose | −3.53 (0.674) | −3.75 (0.755) | −0.23 | (−2.26, 1.80) | 0.8237 |
| Day 22 | −2.16 (0.612) | −2.93 (0.691) | −0.77 | (−2.62, 1.08) | 0.4105 |
| Day 29 | −2.22 (0.615) | −2.81 (0.699) | −0.59 | (−2.45, 1.28) | 0.5331 |
| Day 42 | −3.14 (0.623) | −1.17 (0.673) | 1.96 | (0.12, 3.80) | 0.0368 |

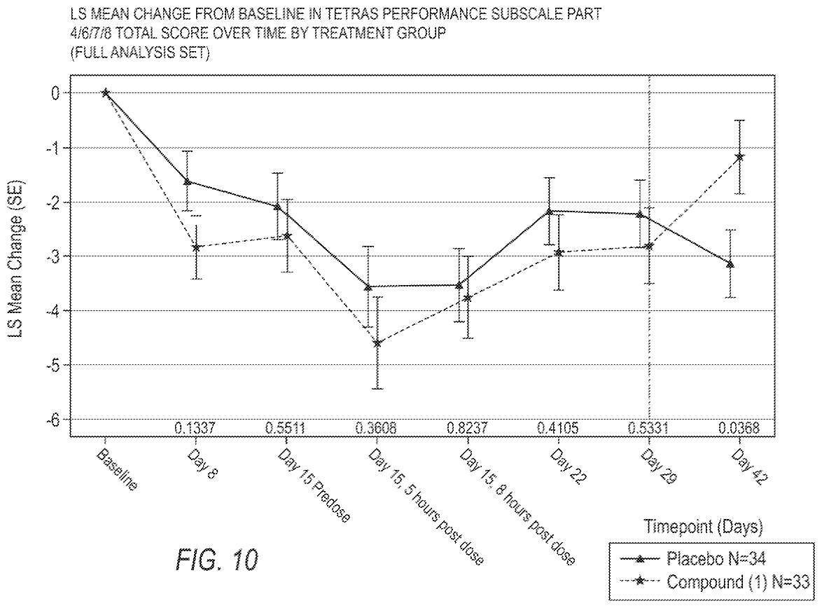

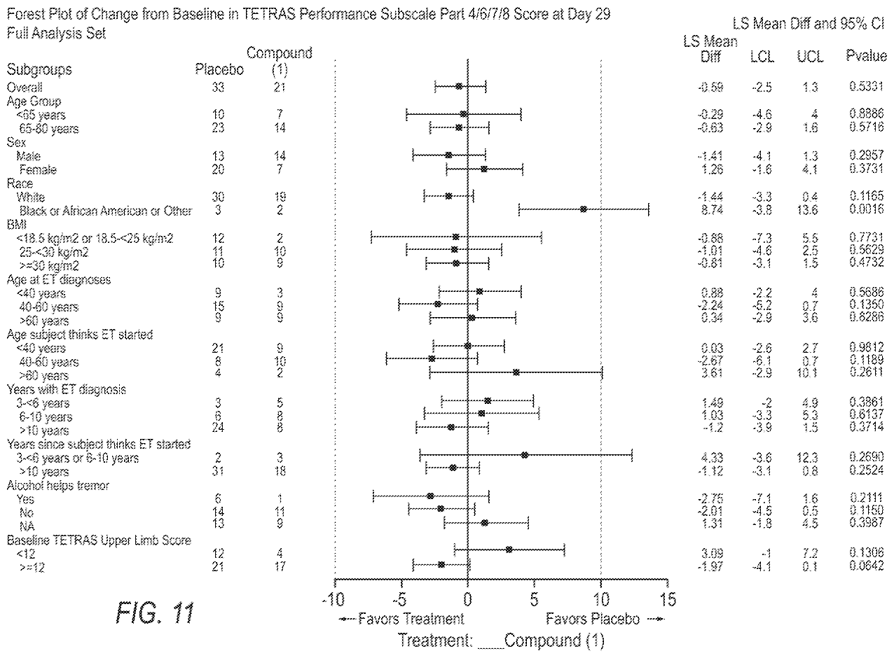

For the Full Analysis Set, LS Mean change from baseline in TETRAS performance subscale part 4/6/7/8 total score over time by treatment group is shown in FIG. 10. A forest plot of change from baseline in TETRAS performance subscale part 4/6/7/8 total score at day 29 is shown in FIG. 11.

### Safety

### Adverse Events

33/34 (97.1%) reported treatment-emergent adverse events (TEAEs) in the Compound 1 group compared to 20/35 (57.1%) in the placebo group.

No deaths were reported during the study.

11/34 (32.4%) participants reported severe TEAEs in the Compound 1 group and 2/35 (5.7%) reported severe TEAEs in the placebo group.

There were 4 participants that experienced severe adverse events (SAEs):

One participant reported a mental status changes (Compound 1 group, severe, occurred on Day 5, resolved on Day 7, related, leading to treatment discontinuation and study discontinuation on 60 mg).

One participant reported a mental status changes (Compound 1 group, severe, occurred on Day 12, resolved on Day 17, related, leading to treatment discontinuation on 60 mg).

One participant (Compound 1 group) reported 3 SAEs: dehydration (mild, occurred on Day 6, resolved on Day 8, unrelated, drug interrupted); headache (moderate, occurred on Day 6, resolved on Day 7, unrelated, drug interrupted); and transient ischaemic attack (mild, occurred on Day 6, resolved on Day 8, unrelated, drug interrupted). This participant had dose reduced to 45 mg on Day 11, further reduced to 30 mg on Day 25, and completed the treatment.

One participant reported angina pectoris and dyspnoea (placebo group, occurred on Day 8, not related).

21/34 (61.8%) reported TEAEs resulting in treatment dose reduction in the Compound 1 group; 1/35 reported TEAEs resulting in treatment dose reduction in the placebo group.

9/34 (26.5%) reported TEAEs leading to treatment discontinuation in the Compound 1 group and 0 reported TEAEs leading to treatment discontinuation in the placebo group. For those 9 participants, 4 discontinued 60 mg from day 5 to day 22, 1 discontinued 45 mg on day 14, and 4 discontinued 30 mg from day 5 to day 15.

4/34 (11.8%) reported TEAEs leading to study discontinuation in the Compound 1 group with 0 reports in the placebo group.

Overall, patients were randomized 1:1 to receive Compound 1 (60 mg) or matched placebo once daily in the morning. The trial evaluated treatment of Compound 1 at the higher end of the dose range and the daily dose could be down-titrated to 45 mg or 30 mg if 60 mg was not well tolerated. Down-titration of dose occurred in 62% of patients who received Compound 1 and discontinuations were noted in 3800 of patients receiving Compound 1. The most common TEAEs that occurred in ≥10% of patients in Compound 1 treatment group and at a rate at least twice as high as that of patients in the placebo group were: somnolence 680%; dizziness 38%; balance disorder 15%; diplopia 12%; dysarthria 12%; and gait disturbance 12%.

TABLE 12
Most frequently reported TEAEs observed in at least 5%
of participants in any treatment group (Safety Set).

| | | |
|---|---|---|
| | Placebo | Cmpd 1 |
| TEAE | (N = 35) | (N = 34) |

| | | | | |
|---|---|---|---|---|
| Somnolence | 2 | (5.7%) | 23 | (67.6%) |
| Dizziness | 4 | (11.4%) | 13 | (38.2%) |
| Fatigue | 4 | (11.4%) | 5 | (14.7%) |

| | | | |
|---|---|---|---|
| Balance disorder | 0 | 5 | (14.7%) |
| Diplopia | 0 | 4 | (11.8%) |
| Dysarthria | 0 | 4 | (11.8%) |
| Gait disturbance | 0 | 4 | (11.8%) |

| | | | | |
|---|---|---|---|---|
| Disturbance in attention | 3 | (8.6%) | 3 | (8.8%) |

| | | | |
|---|---|---|---|
| Myoclonus | 0 | 3 | (8.8%) |

| | | | | |
|---|---|---|---|---|
| Headache | 6 | (17.1%) | 2 | (5.9%) |
| Diarrhoea | 3 | (8.6%) | 2 | (5.9%) |
| Paraesthesia | 2 | (5.7%) | 2 | (5.9%) |

| | | | |
|---|---|---|---|
| Lethargy | 0 | 2 | (5.9%) |
| Mental status changes | 0 | 2 | (5.9%) |
| Speech disorder | 0 | 2 | (5.9%) |

| | | | | |
|---|---|---|---|---|
| Coordination abnormal | 3 | (8.6%) | 1 | (2.9%) |
| Asthenia | 2 | (5.7%) | 1 | (2.9%) |
| Insomnia | 2 | (5.7%) | 1 | (2.9%) |
| Urinary tract infection | 2 | (5.7%) | 1 | (2.9%) |

| | | | |
|---|---|---|---|
| Irritability | 2 | (5.7%) | 0 |
| Myalgia | 2 | (5.7%) | 0 |

The following TEAEs were reported in at least 2 participants who received Compound 1 but not reported by participants received placebo: balance disorder (5 participants), diplopia (4 participants), dysarthria (4 participants), gait disturbance (4 participants), myoclonus (3 participants), lethargy (2 participants), mental status changes (2 participants), speech disorder (2 participants).

### Pharmacokinetic (PK) Concentration:

Compound 1 median mean plasma concentrations over time among treatment completers is shown in Table 13 below. Plasma concentration was measured pre-dose (C_{trough}) on Days 8 and 29. The median plasma concentrations were calculated for all treatment completers irrespective of ending dose group (e.g., 30 mg, 45 mg, or 60 mg).

TABLE 13
Compound 1 plasma concentration over time irrespective
of ending dose among treatment completers.

Compound 1 Plasma Concentration
(ng/mL)

| | Day 8 | Day 29 |
|---|---|---|
| | (N = 28) | (N = 23) |
| Median | 203 | 294 |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A method of treating essential tremor in a subject in need thereof, comprising administering to the subject a dose of from about 5 mg to about 80 mg of Compound 1 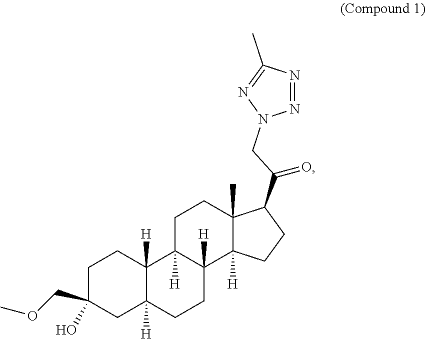
or a dose equivalent of a pharmaceutically acceptable salt thereof, at least once per day.

2. The method of claim 1, wherein a dose of from about 50 mg to about 70 mg of Compound 1, or a dose equivalent of a pharmaceutically acceptable salt thereof, is administered to the subject at least once per day.

3. The method of claim 1, wherein a dose of from about 10 mg to about 30 mg of Compound 1, or a dose equivalent of a pharmaceutically acceptable salt thereof, is administered to the subject at least once per day.

4. The method of claim 1, wherein a dose of from about 20 mg to about 40 mg of Compound 1, or a dose equivalent of a pharmaceutically acceptable salt thereof, is administered to the subject at least once per day.

5. The method of claim 1, wherein a dose of about 10 mg, about 15 mg, about 30 mg, about 45 mg, or about 60 mg of Compound 1, or a dose equivalent of a pharmaceutically acceptable salt thereof, is administered to the subject at least once per day.

6. The method of claim 1, wherein a dose of about 60 mg of Compound 1, or a dose equivalent of a pharmaceutically acceptable salt thereof, is administered to the subject at least once per day.

7. The method of claim 1, wherein a dose of about 45 mg of Compound 1, or a dose equivalent of a pharmaceutically acceptable salt thereof, is administered to the subject at least once per day.

8. The method of claim 1, wherein a dose of about 30 mg of Compound 1, or a dose equivalent of a pharmaceutically acceptable salt thereof, is administered to the subject at least once per day.

9. The method of claim 1, wherein a dose of about 15 mg of Compound 1, or a dose equivalent of a pharmaceutically acceptable salt thereof, is administered to the subject at least once per day.

10. A method of treating essential tremor in a subject in need thereof, comprising administering to the subject a therapeutically effective amount of Compound 1 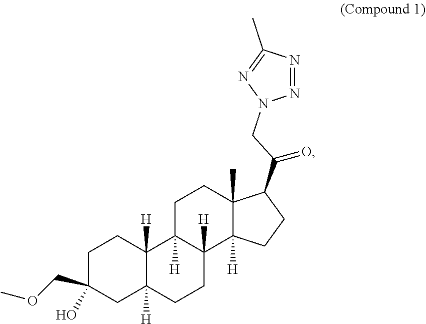
or a pharmaceutically acceptable salt thereof,
wherein the subject experiences a reduction in tremor amplitude of at least about 5% following treatment onset.

11. The method of any one of claims 1-10, wherein the subject experiences a reduction in tremor amplitude of at least about 15% following treatment onset.

12. The method of claim 11, wherein the subject experiences a reduction in tremor amplitude of at least about 30% following treatment onset.

13. The method of claim 12, wherein the subject experiences a reduction in tremor amplitude of at least about 50% following treatment onset.

14. The method of claim 13, wherein the subject experiences a reduction in tremor amplitude of at least about 75% following treatment onset.

15. The method of any one of claims 1-14, wherein the subject experiences a reduction in tremor amplitude about 8 days following treatment onset.

16. The method of any one claims 1-15, wherein the subject experiences a reduction in tremor amplitude about 28 days following treatment onset.

17. The method of any one of claims 1-16, wherein the subject has an initial The Essential Tremor Assessment Scale (TETRAS) performance subscale part 4 upper limb tremor total score of at least about 10 or greater prior to treatment onset.

18. The method of any one of claims 1-16, wherein the subject has an initial TETRAS performance subscale part 4 upper limb tremor total score of at least about 4 prior to treatment onset.

19. The method of any one of claims 1-16 or 18, wherein the subject has an initial TETRAS performance subscale part 4 upper limb tremor total score of at least about 6 prior to treatment onset.

20. The method of any one of claims 1-16, 18, or 19, wherein the subject has an initial TETRAS performance subscale part 4 upper limb tremor total score of at least about 8 prior to treatment onset.

21. The method of any one of claims 1-16 or 18-20, wherein the subject has an initial TETRAS performance subscale part 4 upper limb tremor total score of at least about 10 prior to treatment onset.

22. The method of any one of claims 1-16 or 18-21, wherein the subject has an initial TETRAS performance subscale part 4 upper limb tremor total score of at least about 12 prior to treatment onset.

23. A method of treating essential tremor in a subject in need thereof, comprising administering to the subject a therapeutically effective amount of Compound 1 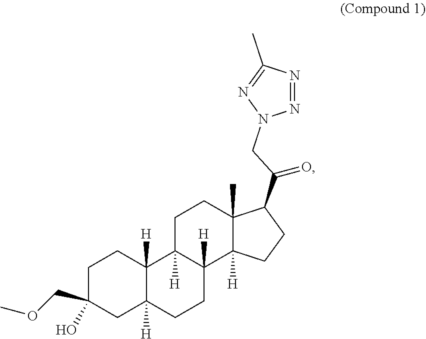
or a pharmaceutically acceptable salt thereof,
wherein the subject experiences a reduction in TETRAS activities of daily living (ADL) subscale total score following treatment onset.

24. The method of claim 23, wherein the subject experiences a reduction in TETRAS ADL subscale total score of at least about 5% following treatment onset.

25. The method of claims 23 or 24, wherein the subject experiences a reduction in TETRAS ADL subscale total score of at least about 10% following treatment onset.

26. The method of any one of claims 23-25, wherein the subject experiences a reduction in TETRAS ADL subscale total score of at least about 15% following treatment onset.

27. The method of any one of claims 23-26, wherein the subject experiences a reduction in TETRAS ADL subscale total score of at least about 20% following treatment onset.

28. The method of any one of claims 23-27, wherein the subject experiences the reduction in TETRAS ADL subscale total score about 8 days following treatment onset.

29. The method of any one of claims, 23-28, wherein the subject experiences a reduction in TETRAS ADL subscale total score about 28 days following treatment onset.

30. The method of any one of claims 23-29, wherein the subject has an initial TETRAS ADL subscale total score of at least 12 prior to treatment onset.

31. The method of any one of claims 23-29, wherein the subject has an initial TETRAS ADL subscale total score of at least about 20 prior to treatment onset.

32. The method of any one of claims 23-29 or 31, wherein the subject has an initial TETRAS ADL subscale total score of at least about 24 prior to treatment onset.

33. The method of any one of claims 23-29, 31, or 32, wherein the subject has an initial TETRAS ADL subscale total score of at least about 28 prior to treatment onset.

34. The method of any one of claims 23-33, wherein the subject is administered a dose of from about 5 mg to about 80 mg of Compound 1 or a dose equivalent of a pharmaceutically acceptable salt thereof, at least once per day.

35. The method of claim 36, wherein the subject is administered a dose of from about 10 mg to about 70 mg of Compound 1 or a dose equivalent of a pharmaceutically acceptable salt thereof, at least once per day.

36. The method of claim 36, wherein the subject is administered a dose of from about 50 mg to about 70 mg of Compound 1 or a dose equivalent of a pharmaceutically acceptable salt thereof, at least once per day.

37. The method of claim 36, wherein the subject is administered a dose of from about 10 mg to about 30 mg of Compound 1 or a dose equivalent of a pharmaceutically acceptable salt thereof, at least once per day.

38. The method of claim 36, wherein the subject is administered a dose of about 10 mg, about 15 mg, about 30 mg, about 45 mg, or about 60 mg of Compound 1 or a dose equivalent of a pharmaceutically acceptable salt thereof, at least once per day.

39. The method of claim 38, wherein the subject is administered a dose of about 60 mg of Compound 1 or a dose equivalent of a pharmaceutically acceptable salt thereof, at least once per day.

40. The method of claim 36, wherein the subject is administered a dose of from about 30 mg to about 50 mg of Compound 1 or a dose equivalent of a pharmaceutically acceptable salt thereof, at least once per day.

41. The method of claim 40, wherein the subject is administered a dose of about 45 mg of Compound 1 or a dose equivalent of a pharmaceutically acceptable salt thereof, at least once per day.

42. The method of claim 36, wherein the subject is administered a dose of from about 20 mg to about 40 mg of Compound 1 or a dose equivalent of a pharmaceutically acceptable salt thereof, at least once per day.

43. The method of any one of claims 1-42, wherein Compound 1, or the pharmaceutically acceptable salt thereof, is administered once per day.

44. The method of any one of claims 1-42, wherein Compound 1, or a pharmaceutically acceptable salt thereof, is administered at least once per day for at least about 28 days.

45. The method of any one of claims 1-44, wherein the subject has a plasma concentration of Compound 1 of at least about 175 ng/mL about 8 days following treatment onset.

46. The method of any one of claims 1-45, wherein the subject has a plasma concentration of Compound 1 of from about 200 ng/mL to about 250 ng/mL about 8 days following treatment onset.

47. The method of any one of claims 1-46, wherein the subject has a plasma concentration of Compound 1 of at least about 210 ng/mL about 15 days following treatment onset.

48. The method of any one of claims 1-47, wherein the subject has a plasma concentration of Compound 1 of from about 210 ng/mL to about 450 ng/mL about 15 days following treatment onset.

49. The method of any one of claims 1-48, wherein the subject has a plasma concentration of Compound 1 of at least about 230 ng/mL about 22 days following treatment onset.

50. The method of any one of claims 1-49, wherein the subject has a plasma concentration of Compound 1 of from about 230 ng/mL to about 390 ng/mL about 22 days following treatment onset.

51. The method of any one of claims 1-50, wherein the subject has a plasma concentration of Compound 1 of greater than about 250 ng/mL about 29 days following treatment onset.

52. The method of any one of claims 1-51, wherein Compound 1 or the pharmaceutically acceptable salt thereof is administered orally.

53. The method of any one of claims 1-52, wherein Compound 1 or the pharmaceutically acceptable salt thereof is administered as a tablet or capsule.

54. A method of treating essential tremor in a subject in need thereof, comprising administering to the subject an initial dose of from about 50 mg to about 80 mg of Compound 1 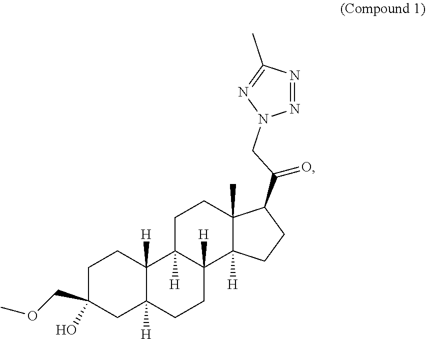
or a dose equivalent of a pharmaceutically acceptable salt thereof,
wherein the initial dose is administered at least once per day.

55. The method of claim 54, wherein the initial dose is administered from onset of treatment for a duration of from about 1 to about 30 days.

56. The method of claim 55, wherein the initial dose is administered from onset of treatment for a duration of about 28 days.

57. The method of any one of claims 54-56, wherein the initial dose is from about 55 mg to about 70 mg of Compound 1 or a dose equivalent of a pharmaceutically acceptable salt thereof.

58. The method of any one of claims 54-57, comprising administering to the subject a second dose comprising from about 25 mg to about 50 mg of Compound 1, or a dose equivalent of a pharmaceutically acceptable salt thereof, wherein the second dose is administered on the day following the last administration of the initial dose.

59. The method of claim 58, wherein the second dose comprises from about 30 mg to about 45 mg of Compound 1, or a dose equivalent of the pharmaceutically acceptable salt thereof.

60. The method of claim 58 or claim 59, wherein the second dose comprises from about 25 mg to about 35 mg of Compound 1, or a dose equivalent of the pharmaceutically acceptable salt thereof.

61. The method of any one of claims 58-60, wherein the second dose is administered for a duration of about 60 days or less.

62. The method of any one of claims 58-61, comprising administering to the subject a third dose comprising from about 5 mg to about 24 mg of Compound 1, or a dose equivalent of the pharmaceutically acceptable salt thereof, wherein the third dose is administered on the day following the last administration of the second dose.

63. The method of claim 62, wherein the third dose comprises from about 5 mg to about 20 mg of Compound 1, or a dose equivalent of the pharmaceutically acceptable salt thereof.

64. The method of claim 62 or claim 63, wherein the third dose comprises from about 7.5 mg to about 17.5 mg of Compound 1, or a dose equivalent of the pharmaceutically acceptable salt thereof.

65. The method of any one of claims 54-64, wherein Compound 1, or the pharmaceutically acceptable salt thereof, is administered orally.

66. The method of any one of claims 54-65, wherein Compound 1, or the pharmaceutically acceptable salt thereof, is administered as a tablet or capsule.

67. A method of treating essential tremor in a subject in need thereof, comprising administering to the subject a therapeutically effective amount of Compound 1 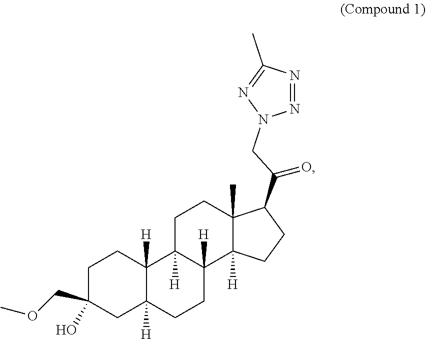
or a pharmaceutically acceptable salt thereof, wherein the subject has a plasma concentration of Compound 1 of from about 100 ng/mL to about 450 ng/mL about 8 days following treatment onset.

68. The method of claim 67, wherein the subject has a plasma concentration of Compound 1 of from about 150 ng/mL to about 400 ng/mL about 8 days following treatment onset.

69. The method of claim 68, wherein the subject has a plasma concentration of Compound 1 of from about 175 ng/mL to about 400 ng/mL about 8 days following treatment onset.

70. The method of claim 69, wherein the subject has a plasma concentration of Compound 1 of from about 175 ng/mL to about 250 ng/mL about 8 days following treatment onset.

71. The method of claim 69, wherein the subject has a plasma concentration of Compound 1 of from about 200 ng/mL to about 250 ng/mL about 8 days following treatment onset.

72. A method of treating essential tremor in a subject in need thereof, comprising administering to the subject a therapeutically effective amount of Compound 1 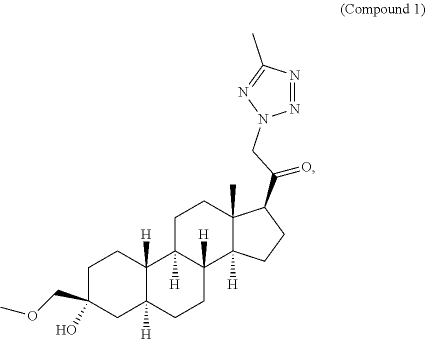
or a pharmaceutically acceptable salt thereof, wherein the subject has a plasma concentration of Compound 1 of from about 210 ng/mL to about 475 ng/mL about 15 days following treatment onset.

73. The method of claim 72, wherein the subject has a plasma concentration of Compound 1 of from about 210 ng/mL to about 450 ng/mL about 15 days following treatment onset.

74. The method of claim 72, wherein the subject has a plasma concentration of Compound 1 of from about 210 ng/mL to about 350 ng/mL about 15 days following treatment onset.

75. The method of claim 72, wherein the subject has a plasma concentration of Compound 1 of from about 210 ng/mL to about 280 ng/mL about 15 days following treatment onset.

76. The method of claim 72, wherein the subject has a plasma concentration of Compound 1 of from about 320 ng/mL to about 420 ng/mL about 15 days following treatment onset.

77. The method of claim 72, wherein the subject has a plasma concentration of Compound 1 of from about 275 ng/mL to about 490 ng/mL about 15 days following treatment onset.

78. A method of treating essential tremor in a subject in need thereof, comprising administering to the subject a therapeutically effective amount of Compound 1 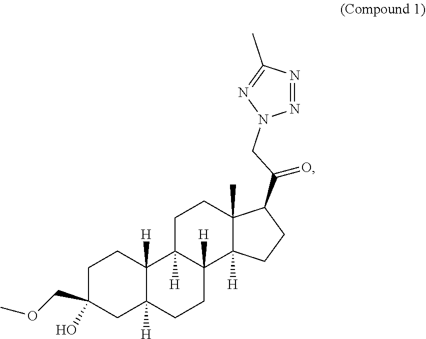
or a pharmaceutically acceptable salt thereof, wherein the subject has a plasma concentration of Compound 1 of from about 230 ng/mL to about 390 ng/mL about 22 days following treatment onset.

79. The method of any one of claims 66-78, wherein Compound 1, or the pharmaceutically acceptable salt thereof, is administered orally.

80. The method of any one of claims 66-79, wherein Compound 1, or the pharmaceutically acceptable salt thereof, is administered as a tablet or capsule.

81. The method of any one of claims 66-80, wherein the plasma concentration is C_{trough}.
